(19) United States
(12) Patent Application Publication
(10) Pub. No.: US 2021/0115158 A1
(43) Pub. Date: Apr. 22, 2021

(54) Synthetic Particle Antibody Compositions And Uses Thereof

(71) Applicant: Georgia Tech Research Corporation, Atlanta, GA (US)

(72) Inventors: Krishnendu Roy, Atlanta, GA (US); Jiaying Liu, Atlanta, GA (US); Randall Toy, Atlanta, GA (US)

(21) Appl. No.: 16/500,289

(22) PCT Filed: Apr. 3, 2018
(86) PCT No.: PCT/US2018/025827

§ 371 Date: Oct. 2, 2019

Related U.S. Application Data
(60) Provisional application No. 62/480,717, filed on Apr. 3, 2017.

(51) Int. Cl. C07K 16/46; G01N 33/533; G01N 33/541; G01N 33/561; C07K 1/26; G01N 33/534

(52) U.S. Cl. CPC C07K 16/468; G01N 33/533; G01N 33/541; G01N 2800/24; C07K 1/26; G01N 33/534; C07K 2317/31; G01N 33/561

(57) ABSTRACT

The invention is directed to a synthetic particle antibody comprising a bi-functional particle framework, such as for example and not limitation, a Janus micro- or nanoparticle, wherein one side of the bi-functional particle comprises targeting ligands (such as for example and not limitation, a protein, a peptide, an aptamer, and/or fragments thereof, wherein the at least one targeting ligand has the ability to specifically bind to a desired cell or tissue type in a subject's body) and the other side of the bi-functional particle comprises immune-activating ligands (such as for example and not limitation, fragments of the Fc portion of antibodies, immune-activating peptides, immune-activating aptamers, and other proteins, peptides or nucleic acids that mimic the structure and/or function of the Fc portion of antibodies).

### CROSS-REFERENCE TO RELATED APPLICATIONS

This application claims priority to U.S. Provisional Application No. 62/480,717, filed on 3 Apr. 2017, the disclosure of which is herein incorporated by reference in its entirety.

### BACKGROUND OF THE DISCLOSURE

### 1. Field of the Disclosure

Embodiments of the present disclosure relates generally to synthetic antibodies, and more specifically to synthetic antibodies comprising a bi-functional particle framework, such as for example and not limitation, a Janus micro- or nanoparticle, wherein one side of the bi-functional particle comprises targeting ligands and the other side of the bi-functional particle comprises immune-activating ligands (such as for example and not limitation, fragments of the Fc portion of antibodies, immune-activating peptides, immune-activating aptamers, and other proteins, peptides or nucleic acids that mimic the structure and/or function of the Fc portion of antibodies).

### 2. Background

Monoclonal antibodies (mAbs) are a family of proteins typically secreted by plasma B cells upon exposure to pathogens. mAbs consist of two heavy chains and two light chains, forming two sub-domains: the Fab domain and the Fc domain. Fab domains are responsible for binding onto specific antigen molecules (via a tertiary structure of polypeptides that comprises the complementarity determining regions or CDRs) while Fc domains engage with receptors on the effector cells (innate immune cells, such as macrophages, natural killer (NK) cells and polymorphonuclear leukocytes) to trigger immune responses. In immunotherapies, these therapeutic proteins function by reducing cell proliferation/inducing tumor cell apoptosis or by eliciting complement activation as well as antibody dependent cellular cytotoxicity, and facilitating the T cell immune response through blockade of immune-suppressive pathways (see, e.g., Scott A M et al 2012).

Monoclonal antibody treatment for cancer has been developed for over three decades and has proved its efficacy in a number of hematological malignancies and solid tumors (see, e.g., Cai 2017, Chames et al. 2009, Anon 2016). Generally, monoclonal antibody therapy for cancer can be divided into two categories: one directly eliminates the tumor cells by antibody-dependent immune responses; the other type modulates the immune factors in tumor microenvironments. The success of the first type of antibody-based therapies relies on tumor-associated antigens (TAAs), which are a group of proteins or molecules that are either selectively expressed, mutated or overexpressed on the surface of malignant cells compared to normal tissues. Antibodies in this category are generally directed to specific TAAs, such as CD20 over-expressed on malignant B cells for treating B cell non-Hodgkin's lymphomas, HER2 for treating aggressive breast cancer and a fraction of ovary and stomach tumors, CD52 in chronic lymphocytic leukemia and J591 for PSMA in prostate carcinoma.

In cases where the cancer does not express a specific or distinct TAA, the second category of antibody-based therapy can prove useful. Rather than directly killing or clearing the malignant cells, the second type of antibody depletes immunosuppressive factors or regulatory immune cells that blunt or block the body's immune responses to cancers and/or tumors, and thus restores the immune system's attack against those cells.

Though monoclonal antibody therapy has had some clinical success, there are obstacles in cancer treatment that have not been overcome by mAbs. Notably, two major inhibitors of the immune response do not have TAAs suitable for mAb therapy. Myeloid derived suppressor cells (MDSCs), which are a heterogeneous population of cells consisting of myeloid progenitors, immature macrophages, immature granulocytes, and immature DCs, accumulate in tumors, spleen and blood, where they secrete various cytokines (e.g., IL-10, TGF-β), enzymes and reactive species to (i) inhibit the proliferation and activation of T effector cells, (ii) regulate the cytokine production of macrophages and (iii) impair the function of natural killer cells. Regulatory T cells are required for protection against autoimmune diseases, which suppress the reactivity of anti-tumor T cells. Thus, inhibition of MDSC and regulatory T cell activity/function is one factor that is necessary to improve the outcome of anti-tumor monoclonal antibody-based therapies.

Another factor mitigating against mAbs lies in their production and application (see, e.g., Anon 2017; Bru et al. 2015; Schirrmann & Hust 2016, Shaughnessy 2012). A typical process to acquire a new type of mAb involves immunization in animals (transgenic or non-transgenic) with antigens, the isolation of antibody-producing B cells, hybridoma production, the selection of high binders by screening, cloning, mAb production in single cell line, purification and validation. This is a long, complex process that usually lasts months and requires large amount of labor and high production costs. Once produced, the mAb must also be “humanized” to avoid clearance by the immune system, as well as to be able to activate antibody-dependent immunity in human patients. While scFv phage display screening techniques have been developed to bypass production in animals, these new methods require putting the fragments identified by phage display together into a complete antibody, as well as an in vitro system to synthesize and post-translationally modify the antibodies. Regarding application, many targets of interest do not have known surface antigens suitable for mAb therapies. The molecular size and their interaction with the neonatal Fc receptors (FcRn) enable long circulation time of mAbs, but hamper their deep penetration into tissues of interest, including solid tumors, which are characterized by heterogeneous and tortuous vasculatures, a high interstitial fluid pressure, and a high viscosity of the tumor blood supply (see, e.g., Chames et al. 2009). As a result, the therapeutic potency of mAbs in solid tumors is limited. In addition, mAbs have to compete with patients' IgG for binding onto the effective Fc receptors, which further increases the dosage required to achieve satisfactory therapeutic responses. For these reasons, antibodies are more suited to treating hematological cancers than solid tumors, as their pharmacokinetics give them a long serum half-life but poor penetration into solid tumors and poor retention in those tissues (which make up the majority of cancers).

To overcome the challenges of mAb therapies, much research has been done in the field of synthetic or artificial antibodies. A few types of artificial antibodies have been developed so far (e.g., nanobody, minibody, and peptibody) (see, e.g., Scott A M et al 2012, Dorresteijn, B. 2015, and Holliger, P. et al 2005), most of which are peptide/protein replacements for the functional parts of monoclonal antibodies (see, e.g., Wang & Fan 2016; Mazor et al. 2017; Torchia et al. 2016). Nanobodies are single domain antibodies retrieved from an immune library of camelidae (see, e.g., Wang, Y. et al 2016). Due to their small size (2.5 nm in diameter), nanobodies are better able to penetrate tissue than conventional mAbs (see, e.g., Dorresteijn, B. 2015, Wang, Y. et al 2016, De Meyer T et al 2014, and Danquah, W. et al 2016). Nanobodies have been successfully used in solid tumor treatment, targeted drug delivery and bioimaging. However, the small size of the nanobodies generally causes them to be rapidly cleared by the renal system and to accumulate in the kidney, making them less favored for use in clinical applications (see, e.g., Danquah, W. et al 2016). The minibody, a bivalent single-chain antibody composed of scFv domains and the CH3 fragment of Fc linked by amino acid linkers, has achieved higher tumor-to-blood ratio than intact immunoglobins (IgGs), but the exposed amino acid linkers can lead to increased protease degradation and thus rapid loss of function (see, e.g., Holliger P. et al 2005 and Secchiero, P. et al 2009). Peptibodies, which consist of two copies of synthetic peptide ligands for target binding covalently linked to the amino terminus of a recombinant IgG Fc domain, have also exhibited potent biological activity and good targeting specificity (see, e.g., Wu, B. et al 2014). However, despite improved tumor accumulation, the current artificial antibodies suffer from the same production and cost problem as mAbs because they still rely on eukaryotic cell expression of modified or unmodified genetic constructs (see, e.g., Wesolowski, J. et al 2009 and Scott A M et al 2012).

An improved synthetic antibody is therefore needed. This synthetic antibody should allow simpler and more flexible design of the antibodies, as well as an optimized synthetic procedure, that results in antibodies that are functional alternatives to current antibody therapies and applications (in both research and diagnostic areas). Specifically, these synthetic antibodies should utilize synthetic peptides, aptamers or other synthetic targeting and effector molecules on nanoparticles to produce fully synthetic particle antibodies that have offer multi-valency and lower production costs and shorter production time than conventional mAbs. These synthetic particle antibodies can be used in antibody-based therapies for cancer with great translational potential, because these synthetic particle antibodies can bind to specific antigens, including TAAs, and trigger antibody-dependent cytotoxicity in the same way as conventional mAbs. Finally, the synthetic particle antibodies should be capable of both (i) multivalent binding to a target site and (ii) multivalent activation of the innate immune system by using a bi-functional particle to display multiple targeting ligands on one side of the particle's surface and multiple innate immune cell activating moieties on the opposite side. The targeting ligands may be identified by various high-throughput screening/engineering methods, such as phage display biopanning techniques, aptamer screening, and structural mimetic engineering approaches, and then synthesized in large scale. On the one hand, high valency leads to increased binding avidity and selectivity to targets. (Montet et al. 2006; Safenkova et al. 2010; Popov et al. 2011). On the other hand, the activation of antibody-dependent responses relies on the clustering of Fc receptors on the effector cells such as macrophages and natural killer cells by multiple IgG-Fcs. Presentation of multiple Fc-mimicking ligands on the synthetic nanoparticle antibodies increases the crosslinking of Fc receptors on the surface of cells, which triggers a high magnitude of Fc receptor-mediated intracellular signaling and potentially result in stronger activation, phagocytosis and pro-inflammatory cytokine/reactive species release. (Ortiz et al. 2016; Zhang et al. 2010). No eukaryotic machinery is needed to produce the synthetic particle antibodies. Instead, the synthetic particle antibodies are produced through conjugation of unique targeting and immune-activating ligands onto the bi-functional particle.

Further, these synthetic particle antibodies may have potential advantages over conventional mAbs in terms of therapeutic application: deeper tissue penetration, targeting of previously inapplicable cells for mAbs due to lack of known targeting antigens, stronger immune-activation due to multivalency, and an easily adaptable platform to generate new types of synthetic particle antibodies by varying the target-binding peptides.

The synthetic particle antibodies also have advantages over other types of synthetic antibodies. References such as, e.g., U.S. Pat. No. 8,241,651, WO 2011/050105, U.S. Pat. Nos. 7,767,017, 7,947,772, and 7,871,622 all describe multi-phasic nanostructures which have been developed through polymer-based or fusion protein-based strategies. Each of these structures has at least two chemically distinct exposed surfaces and thus is able to conjugate and deliver a variety of binding ligands or therapeutic agents at the same time. Compared to these structures, the synthetic particle antibodies of the disclosure utilize bi-functional particles with ligands with specific, multi-valent immune-activating and targeting ability. In contrast to these multi-phasic nanostructures, which were designed for the delivery of drugs and diagnostic agents, an application of the synthetic particle antibodies of the disclosure is to deplete biomolecules and cell targets through activation of antibody dependent cytotoxicity and/or phagocytosis. References such as, e.g., EP 2564203 and WO 2012/054564 describe antibody-nanoparticle conjugates that block specific receptor-ligand interactions or detect targeted molecules. Again, the use of bi-functional particles in synthetic antibodies of the disclosure enables multivalent presentation of target ligands on one face for targeting with high binding avidity and multivalent presentation of immune-activating ligands on the opposing face to amplify the immune response. References such as, e.g., U.S. Pat. Nos. 8,722,859 and 8,883,162 are directed to the development of multivalent antibody constructs for therapeutic inhibition of molecular signaling pathways in disease treatment. Compared to these designs, the synthetic particle antibodies of the disclosure generally possess more tunable biochemical properties; for example, the targeting face can display a variety of different ligands in combination with Fc-functional domains that mediate immune system activation. Further, the synthetic particle antibodies of the disclosure do not include conjugated biological antibodies; rather, the invented particles are fully synthetic. References such as, e.g., WO 2007/124090 discusses methods to make long-term stable formulations comprising a recombinant protein-engineered therapeutic peptibody. Compared to these methods, the synthetic particle antibodies of the disclosure are boosting the immune response with a multi-valent design to enhance the therapeutic effect while lowering the cost. The invented system is also based on a synthetic organic or inorganic nanoparticle rather than a protein-engineered scaffold. References such as, e.g., U.S. Pat. No. 9,439,966 describe multi-component nanochains that are constructed by connecting nanoparticles made with asymmetric surface chemistry in a controlled fashion, and can also have antibodies can be conjugated to the nanochain, which is distinct from the synthetic particle antibodies of the disclosure. Other references describing nanobodies include, e.g., US 2009/0252681 and U.S. Pat. No. 8,703,131. In contrast to nanobodies, the synthetic particle antibodies of the disclosure can enable enhanced biodistribution through tunability of the particle core and/or the capability to co-deliver alternative therapeutics or contrast agents. Other references describing minibodies include, e.g., US 2011/0268656, U.S. Pat. Nos. 8,772,459, and 5,837,821. In contrast to minibodies, the synthetic particle antibodies of the disclosure include particles that can enable a higher degree of multivalency than the minibody by virtue of the ability to use a particle core of a larger size. Other references describing synthetic antibodies that lack particle cores and thus the advantages of the synthetic particle antibodies described herein include, e.g., U.S. Pat. Nos. 5,770,380, 6,136,313, WO 2008/048970, and US 2004/0018587.

Literature references that describe distinct synthetic nanoparticles include, e.g., Safenkova et al 2010 (discussing the increase of affinity towards specific antigens with size increase of colloidal gold carriers, i.e. with the valency of the conjugates); Soukka et al 2001 (demonstrating that by conjugating monoclonal antibody onto fluorescent, europium (III) nanoparticles, the binding affinity was increased and nonspecific binding was reduced in comparison to antibodies in soluble form); Choi et al 2008 (presenting a surface plasmon resonance based immunosensor using antibody-gold nanoparticle conjugates for antigen detection); Jung et al 2014 (using phage display techniques to identify peptides that have specific binding affinity with selected targets and conjugate the peptides onto nanoparticles to enable targeted delivery of therapeutic agents and showing that with phage-display identified peptides, the effectiveness of DC particulate vaccines was enhanced); Gray et al 2013 (demonstrating that the efficiency of nanoparticle-based delivery of conjugated target-specific peptides can be enhanced through the use of higher affinity peptides selected through phage display or multivalent presentation on the nanoparticle surface); Kaewsaneha et al 2013 (reviewing the state of art of Janus particles and their applications); Tang J et al 2012 (development of a bifunctional microparticle, which presents high densities of different bioactive protein molecules on two hemispheres. This enables a range of capabilities for drug delivery and bioimaging. In contrast to these technologies, the synthetic particle antibodies of the disclosure have two distinct functionalities: one is a target-binding ligand and the other is an immune-activating ligand, enabling the invented particles to perform the function of an antibody instead of a delivery vehicle; Torchia et al 2016 (describing a patient-idiotype-specific peptibodies that can trigger tumor cell phagocytosis by macrophages, which provide a new alternative of lymphoma therapies with less toxicities. Compared to this method, the synthetic particle antibodies of the disclosure are multi-valent, which can augment the patient's immune response); Ortiz et al 2016 (investigating the effect of valency on activation of FcgRs in immune cells and reported the inhibitory function of a construct of three covalent-linked Fc domains); and Tang L et al 2014 (demonstrating the increased capability of tumor penetration by 50 nm nanoparticles in comparison to smaller or larger nanoconjugates).

The synthetic particle antibodies of the disclosure are capable of replacing conventional and currently available synthetic antibodies in antibody-based diagnostic and research applications, and can have improved pharmacokinetics, reduced cost and time of manufacturing, and the possibility of generating enhanced immune system response. It is to such a composition and methods of use that embodiments of the present disclosure are directed.

### BRIEF SUMMARY OF THE DISCLOSURE

As specified in the Background Section, there is a great need in the art to identify technologies for synthetic antibodies and use this understanding to develop novel synthetic antibodies that can replace conventional antibodies in therapeutic, diagnostic and research applications. The present disclosure satisfies this and other needs. Embodiments of the present disclosure relate generally to synthetic antibodies and more specifically to synthetic antibodies comprising a bi-functional particle framework, such as for example and not limitation, a Janus micro- or nanoparticle, wherein one side of the bi-functional particle comprises targeting ligands (such as for example and not limitation, proteins, peptides, aptamers, and/or fragments thereof that have the ability to specifically bind to a desired cell or tissue type in a subject's body) and the other side of the bi-functional particle comprises immune-activating ligands (such as for example and not limitation, fragments of the Fc portion of antibodies, immune-activating peptides, immune-activating aptamers, and other proteins, peptides or nucleic acids that mimic the structure and/or function of the Fc portion of antibodies). Synthetic particle antibodies of the disclosure generally have lower production costs and shorter production time than conventional mAbs. These synthetic particle antibodies can be used in antibody-based therapies for cancer with great translational potential, because these synthetic particle antibodies can bind to specific antigens, including TAAs, and trigger antibody-dependent cytotoxicity in the same way as conventional mAbs. Finally, the synthetic particle antibodies should be capable of both (i) multivalent binding to a target site and (ii) multivalent activation of the innate immune system by using a bi-functional particle to display multiple targeting ligands on one side of the particle's surface and multiple innate immune cell activating moieties on the opposite side. The targeting ligands may be identified by various high-throughput screening/engineering methods, such as phage display biopanning techniques, aptamer screening, and structural mimetic engineering approaches, and then synthesized in large scale. Further, these synthetic particle antibodies may have potential advantages over conventional mAbs in terms of therapeutic application: deeper tissue penetration, targeting of previously inapplicable cells for mAbs due to lack of TAAs, and an easily adaptable platform to generate new types of synthetic particle antibodies by varying the target-binding peptides. The synthetic particle antibodies of the disclosure are capable of replacing conventional and currently available synthetic antibodies in antibody-based diagnostic and research applications, and can have improved pharmacokinetics, reduced cost and time of manufacturing, and the possibility of generating enhanced immune system response.

In one aspect, the disclosure provides a synthetic particle antibody comprising: (i) a bi-hasic particle core that has two different surface chemistries; (ii) at least one targeting ligand conjugated to one hemisphere of the bi-functional particle core; and (iii) at least one immune-activating ligand conjugated to the opposite hemisphere of the bi-functional particle core.

In an embodiment, the bi-functional particle core comprises a Janus particle.

In another embodiment, the at least one targeting ligand comprises a protein, a peptide, an aptamer, and/or fragments thereof, wherein the at least one targeting ligand has the ability to specifically bind to a desired cell or tissue type in a subject's body.

In yet another embodiment, the at least one immune-activating ligand comprises a fragment of the Fc portion of antibodies, an immune-activating peptide, and/or other proteins or peptides that mimic the structure and/or function of the Fc portion of antibodies.

In an embodiment, the at least one targeting ligand comprises the G3 peptide.

In another embodiment, the at least one immune-activating ligand comprises the Pep33 peptide.

In a related aspect, the disclosure provides a method of treating cancer in a patient in need thereof, the method comprising administering a synthetic particle antibody composition comprising: (i) a bi-functional particle core that has two different surface chemistries; (ii) at least one targeting ligand conjugated to one hemisphere of the bi-functional particle core; and (iii) at least one immune-activating ligand conjugated to the opposite hemisphere of the bi-functional particle core, wherein the at least one targeting ligand has specificity to a target selected from the group consisting of (a) a tumor-associated antigen characteristic of the cancer being treated, and (b) a cell surface molecule expressed by a MDSC or a regulatory T cell.

In a related aspect, the disclosure provides a method of treating an autoimmune disease in a patient in need thereof, the method comprising administering a synthetic particle antibody composition comprising: (i) a bi-functional particle core that has two different surface chemistries; (ii) at least one targeting ligand conjugated to one hemisphere of the bi-functional particle core; and (iii) at least one immune-activating ligand conjugated to the opposite hemisphere of the bi-functional particle core, wherein the at least one targeting ligand has specificity to a target selected from the group consisting of (a) a molecule characteristic of the autoimmune disease being treated, (b) a surface molecule expressed by a cell that is a cause of the autoimmune disease or produces the deleterious symptoms of the disease and (c) a molecule that is implicated as a cause of an effect of the autoimmune disease.

In a related aspect, the disclosure provides a method of treating an infection in a patient in need thereof, the method comprising administering a synthetic particle antibody composition comprising: (i) a bi-functional particle core that has two different surface chemistries; (ii) at least one targeting ligand conjugated to one hemisphere of the bi-functional particle core; and (iii) at least one immune-activating ligand conjugated to the opposite hemisphere of the bi-functional particle core, wherein the infection being treated is selected from the group consisting of bacterial, viral, parasitic, and fungal, and wherein the at least one targeting ligand has specificity to a target selected from the group consisting of (a) an antigen characteristic of the infection being treated, and (b) a cell surface molecule expressed by a MDSC or a regulatory T cell.

In a related aspect, the disclosure provides a method of diagnosing a disease or condition in a subject, the method comprising: (a) obtaining a bodily fluid or tissue sample from the subject; (b) contacting the sample with a synthetic particle antibody composition comprising: (i) a bi-functional particle core that has two different surface chemistries; (ii) at least one targeting ligand conjugated to one hemisphere of the bi-functional particle core; and (iii) at least one immune-activating ligand conjugated to the opposite hemisphere of the bi-functional particle core; (c) determining the presence or absence of an antigen that is characteristic of the disease or condition.

In a related aspect, the disclosure provides a method of performing in vivo imaging in a patient in need thereof, the method comprising: (a) administering a synthetic particle antibody composition comprising: (i) a bi-functional particle core that has two different surface chemistries; (ii) at least one targeting ligand conjugated to one hemisphere of the bi-functional particle core; and (iii) at least one immune-activating ligand conjugated to the opposite hemisphere of the bi-functional particle core; (b) placing the patient in an appropriate imaging machine suitable for contrast imaging; and (c) performing the contrast imaging, wherein the synthetic particle antibody composition comprises a contrast agent comprising iron oxide particles or gold particles.

In a related aspect, the disclosure provides a method of immunoprecipitation, the method comprising: (a) mixing and incubating a sample lysate with the synthetic particle antibody according to claim 1, wherein the synthetic particle antibody is conjugated to an antigen of interest; (b) mixing the sample lysate and synthetic particle antibody with at least one suitable bead for immunoprecipitation; and (c) washing and eluting the sample lysate from the at least one bead.

In a related aspect, the disclosure provides a method of immunohistochemistry, the method comprising: (a) fixing a tissue sample in 4% formaldehyde solution; (b) embedding the fixed tissue sample in either tissue freezing medium or paraffin; (c) slicing the embedded tissue sample in 10-20 um sections; (d) adding an appropriate blocking solution to the sliced tissue section; (e) adding at least one synthetic particle antibody according to claim 1 to the tissue section; (f) adding a secondary antibody that recognizes the immune-activating ligands on the at least one synthetic particle antibody to the tissue section; (g) washing and mounting the tissue sections; and (h) imaging the washed and mounted tissue sections for microscopy.

In a related aspect, the disclosure provides a method for enzyme-linked immunosorbent assay (ELISA), the method comprising: (a) coating a well plate or other substrate with at least one synthetic particle antibody according to claim 1; (b) adding a sample with proteins that are recognized by the targeting ligands on the at least one synthetic particle antibody; (c) adding a secondary antibody that recognizes the immune-activating ligands on the at least one synthetic antibody particle, wherein the secondary antibody can be conjugated to a fluorophore or a tertiary antibody linked to an enzyme; and (d) performing an assay measuring fluorescence from the secondary antibody or absorbance from reaction of the tertiary antibody linked to an enzyme with a substrate.

In a related aspect, the disclosure provides a method of immunoblotting, the method comprising: (a) isolating proteins from tissue samples or cell culture; (b) separating proteins using gel electrophoresis; (c) transferring proteins from the gel to a membrane; (d) blocking the membrane to prevent non-specific interactions with proteins and the at least one synthetic particle antibody; (e) incubating the membrane with the at least one synthetic particle antibody with targeting ligands specific to a protein of interest; (f) rinsing the membrane and adding a secondary antibody that recognizes the immune-activating ligands on the at least one synthetic particle antibody, in which the secondary antibody can be conjugated at least one reporter comprising a fluorophore, a chemiluminescent substrate, a radioactive label, or a tertiary antibody linked to an enzyme; and (g) performing an assay that measures protein levels by methods that are not limited to fluorescence, luminescence, or radiography.

These and other objects, features and advantages of the present disclosure will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIGS. 1A-1B. Exemplary synthetic particle antibodies. FIG. 1A shows multiple embodiments of synthetic particle antibodies according to the disclosure. FIG. 1B depicts a monoclonal antibody.

FIGS. 2A-2C. Exemplary method of preparing synthetic particle antibodies. This non-limiting example depicts a synthetic particle antibody fabrication procedure using solid-phase chemistry. (FIG. 2A) Exemplary fabrication procedure of Janus gold nanoparticles from streptavidin (SA)-modified gold nanoparticles (AuNP). (FIG. 2B) Structure of Janus gold nanoparticles following coating one hemisphere with thiol groups and the other hemisphere with free biotin-binding sites on streptavidin. (FIG. 2C) Modification of Janus gold nanoparticles with targeting ligands and immune-activating ligands.

FIGS. 3A-3B. Validation of bi-functional conformation of the particles. (FIG. 3A) 3 nm biotin-gold nanoprobes bound onto the free biotin-binding sites on the unmodified streptavidin coated gold nanoparticles or the hemisphere with free biotin-binding sites on the bi-functional gold nanoparticles. (FIG. 3B) Diagrammatic representation of the bi-functional particle with surface chemistries to bind biotinylated target ligands (as exemplified by 3 nm biotin-gold nanoprobes) on one side of the particle and thiol groups available to bind immune-activating ligands on the other side.

FIGS. 4A-4B. Validation of the existence and availability of thiol groups for maleimide reactive groups. (FIG. 4A) Particles after conjugation with Alexa Fluor 647-Maleimide dye as an exemplary maleimide-terminated ligand. (FIG. 4B) Diagrammatic representation of the bi-functional particle with thiol groups available for binding.

FIGS. 5A-5B. Validation of peptide modification on Janus gold nanoparticles with fluorescently labeled peptide ligands. (FIG. 5A) Fluorescence intensity of nanoparticles labeled with exemplary targeting ligand G3-biotin. (FIG. 5B) Fluorescence intensity of nanoparticles labeled with exemplary immune-activating ligand Pep33-SMCC.

FIG. 6. Activation of NFkB proinflammatory pathway of RAW Blue macrophages by synthetic particle antibodies. An increase in the absorbance reading indicates an increase in the amount of alkaline phosphatase that is secreted from the RAW Blue macrophages when NFkB is activated. The increase in alkaline phosphatase secretion after synthetic particle antibody (SNAb) treatment thus indicates a higher level of activation of the NFkB pathway, suggesting stronger immune activities of macrophages after treatment with synthetic particle antibodies.

FIGS. 7A-7B. Validation of synthetic particle antibodies binding on cell targets by photoacoustic imaging. (FIG. 7A) Photoacoustic signals increased in the samples of (i) G3 and Pep33-conjugated synthetic particle antibodies or (ii) AuNP-Pep33 treated cell samples, indicating binding of these particles on these cells, possibly by G3-MDSC interaction and Pep33-Fc receptor interaction. (FIG. 7B) Quantification of cells treated with various synthetic particle antibodies.

FIGS. 8A-8B. Killing of myeloid-derived suppressor cells (MDSCs) in splenocyte mixed co-cultures induced by synthetic particle antibodies. (FIG. 8A) Percentage of total MDSCs in the co-cultures following treatment with various synthetic particle antibodies. (FIG. 8B) Percentage of dead MDSCs in the co-cultures following treatment with various synthetic particle antibodies.

FIGS. 9A-9E. In vivo depletion of MDSCs by synthetic particle antibodies in a 4T1 breast cancer murine model. FIGS. 9A-9C show the numbers of total cells in the spleen (FIG. 9A), the percentage of granulocytic MSDCs in the spleen (FIG. 9B), and the percentage of monocytic MSDCs in the spleen (FIG. 9C). FIGS. 9D-9E show the percentage of granulocytic MSDCs relative to total CD11b⁺ cells in blood (FIG. 9D), and the percentage of monocytic MSDCs relative to total CD11b⁺ cells in blood (FIG. 9E).

FIGS. 10A-10C. In vivo distribution of synthetic particle antibodies in lung, liver, spleen, kidney, tumor and blood in a 4T1 breast cancer murine model. (FIG. 10A) Size of non-Janus AuNP-SA and Janus SH-AuNP-SA synthetic particle antibodies as determined by zetasizer. (FIG. 10B) Biodistribution of synthetic particle antibodies in different organs by percentage at different time points after intravenous injection via tail vein in 4T1-breast tumor bearing Balb/c mice. The biodistribution is calculated as the percentage of Au in each organ out of the sum of the amount measured in the six organs, showing relative abundancy of synthetic particle antibodies in each of these organs. (FIG. 10C) Biodistribution of synthetic particle antibodies in different organs by concentration at different time points after intravenous injection via tail vein in 4T1-breast tumor bearing Balb/c mice.

### DETAILED DESCRIPTION OF THE DISCLOSURE

As specified in the Background Section, there is a great need in the art to identify technologies for synthetic antibodies and use this understanding to develop novel synthetic antibodies that can replace conventional antibodies in therapeutic, diagnostic and research applications. The present disclosure satisfies this and other needs. Embodiments of the present disclosure relate generally to synthetic antibodies and more specifically to synthetic antibodies comprising a bi-functional particle framework, such as for example and not limitation, a Janus micro- or nanoparticle, wherein one side of the bi-functional particle comprises targeting ligands (such as for example and not limitation, proteins, peptides, aptamers, and/or fragments thereof that have the ability to specifically bind to a desired cell or tissue type in a subject's body) and the other side of the bi-functional particle comprises immune-activating ligands (such as for example and not limitation, fragments of the Fc portion of antibodies, immune-activating peptides immune-activating aptamers, and other proteins, peptides or nucleic acids that mimic the structure and/or function of the Fc portion of antibodies).

Synthetic particle antibodies of the disclosure generally have lower production costs and shorter production time than conventional mAbs. These synthetic particle antibodies can be used in antibody-based therapies for cancer with great translational potential, because these synthetic particle antibodies can bind to specific antigens, including TAAs, and trigger antibody-dependent cytotoxicity in the same way as conventional mAbs. General methods of producing synthetic particle antibodies of the disclosure include the conjugation of unique binding and activating ligands onto a bi-functional particle, which exhibits two distinct surface chemistries. In some embodiments, the bi-functional particle can be produced as follows: first, unmodified particles are attached to a solid phase resin with a heterobifunctional, reducible crosslinker. Following washing of unbound particles, bound particles are cleaved from the resin using a reducing agent. The resultant particle can exhibit a thiol surface chemistry on one side of the surface and its original surface chemistry on the opposing face. Therefore, unique moieties can be attached to each side of the surface of the bi-functional particle. The simple production procedure of the synthetic particle antibodies takes no longer than several days given all the building blocks available. To further reduce production time, a stock of particles already modified with immune-activating ligands can be prepared and later transformed into a variety of fully functional synthetic nanoparticle antibodies immediately after modification with desired targeting ligands. No eukaryotic machinery is needed to generate these synthetic particle antibodies, and thus the production cost is significantly reduced.

The synthetic particle antibodies should be capable of both (i) multivalent binding to a target site and (ii) multivalent activation of the innate immune system by using a bi-functional particle to display multiple targeting ligands on one side of the particle's surface and multiple innate immune cell activating moieties on the opposite side. The targeting ligands may be identified by various high-throughput screening/engineering methods, such as phage display biopanning techniques, aptamer screening, and structural mimetic engineering approaches, and then synthesized in large scale. Further, these synthetic particle antibodies may have potential advantages over conventional mAbs in terms of therapeutic application: deeper tissue penetration, targeting of previously inapplicable cells for mAbs due to lack of TAAs, and an easily adaptable platform to generate new types of synthetic particle antibodies by varying the target-binding peptides. The synthetic particle antibodies of the disclosure are capable of replacing conventional and currently available synthetic antibodies in antibody-based diagnostic and research applications, and can have improved pharmacokinetics, reduced cost and time of manufacturing, and the possibility of generating enhanced immune system response.

To facilitate an understanding of the principles and features of the various embodiments of the disclosure, various illustrative embodiments are explained below. Although exemplary embodiments of the disclosure are explained in detail, it is to be understood that other embodiments are contemplated. Accordingly, it is not intended that the disclosure is limited in its scope to the details of construction and arrangement of components set forth in the following description or examples. The disclosure is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed disclosure or to imply that certain features are critical, essential, or even important to the structure or function of the claimed disclosure. Rather, these terms are merely intended to highlight alternative or additional features that may or may not be utilized in a particular embodiment of the present disclosure. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present disclosure are intended to be illustrative and not restrictive. Many suitable materials that would perform the same or a similar function as the materials described herein are intended to be embraced within the scope of the disclosure. Such other materials not described herein can include, but are not limited to, materials that are developed after the time of the development of the disclosure, for example. Any dimensions listed in the various drawings are for illustrative purposes only and are not intended to be limiting. Other dimensions and proportions are contemplated and intended to be included within the scope of the disclosure.

As used herein, the term “subject” or “patient” or “individual” refers to mammals and includes, without limitation, domestic animals (e.g., cows, sheep, cats, dogs, and horses), primates (e.g., humans and non-human primates such as monkeys), rabbits, and rodents (e.g., mice and rats). In certain embodiment, the subject is human.

As used herein, the term “combination” of a synthetic particle antibody of the disclosure and at least a second pharmaceutically active ingredient means at least two, but any desired combination of compounds can be delivered simultaneously or sequentially (e.g., within a 24-hour period). It is contemplated that when used to treat various diseases, the compositions and methods of the present disclosure can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy. Administration of a composition according to the disclosure and another therapeutic agent can occur simultaneously in one composition, or simultaneously in different compositions, or sequentially (preferably, within a 24-hour period) in different compositions.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art, nonlimiting embodiments of which are discussed below. An antibody is said to be “capable of binding” a molecule if it is capable of specifically reacting with the molecule to thereby bind the molecule to the antibody.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁; IgG₂, IgG₃, IgG₄, IgA₁; and IgA₂. The heavy chain constant domains that correspond to the different classes of immunoglobulins are called α, δ, ε, γ, and μ, respectively. IgM is the first immunoglobulin expressed during B cell development as a monomer on the surface of B naive cells. The pentameric structure of IgM antibodies makes them efficient at binding antigens with repetitive epitopes (e.g. bacterial capsule, viral capsid) and activation of complement cascade. The IgG, IgE, and IgA antibody isotypes are generated following class-switching during germinal center reaction and provide different effector functions in response to specific antigens. IgG is the most abundant antibody class in the serum and it is divided into 4 subclasses based on differences in the structure of the constant region genes and the ability to trigger different effector functions. Despite the high sequence similarity (90% identical on the amino acid level), each subclass has a different half-life, a unique profile of antigen binding and distinct capacity for complement activation. IgG1 antibodies are the most abundant IgG class and dominate the responses to protein antigens. Impaired production of IgG1 is observed in some cases of immunodeficiency and is often associated with recurrent infections. The IgG responses to bacterial capsular polysaccharide antigens are mediated primarily via IgG2 subclass, and deficiencies in this subclass can result in susceptibility to certain bacterial species. IgG2 represents the major antibody subclass reacting to glycan antigens but IgG1 and IgG3 subclasses have also been observed in such responses, particularly in the case of protein-glycan conjugates. IgG3 is an efficient activator of pro-inflammatory responses by triggering the classical complement pathway. It has the shortest half-life compared to the other IgG subclasses and is frequently present together with IgG1 in response to protein antigens in particular after viral infections. IgG4 is the least abundant IgG subclass in the serum and is often generated following repeated exposure to the same antigen or during persistent infections. IgE antibodies are present at lowest concentrations in peripheral blood but constitute the main antibody class in allergic responses through the engagement of mast cells, eosinophils and Langerhans cells. IgA antibodies are secreted in the respiratory or the intestinal tract and act as the main mediators of mucosal immunity.

The terms “Fc portion”, “Fc fragment”, and/or “Fc ligand” are used interchangeably herein and refer to the C-terminal region of an immunoglobulin heavy chain that contains at least a portion of the constant region. The term includes native sequence Fc fragments and variant Fc fragments. The Fc fragment interacts with cell surface receptors called Fc receptors and some proteins of the complement system, thus allowing antibodies to activate the immune system.

The terms “antigen-binding fragment” or “Fab fragment” refer to the region on an antibody that binds to antigens. It is composed of one constant and one variable domain of each of the heavy and the light chain. The variable domain contains the paratope (the antigen-binding site), comprising a set of complementarity determining regions, at the amino terminal end of the monomer. A Fab fragment is one or more fragments of an antibody that retain the ability to specifically bind to an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂fragment that has two antigen-combining sites and is still capable of cross-linking antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CHI domains; (ii) a F(ab′)₂fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody; and (v) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding portion” of an antibody. The Fab fragment contains the heavy- and light-chain variable domains and also contains the constant domain of the light chain and the first constant domain (CHI) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

As used herein, the term “antigen” refers a molecule capable of inducing an immune response (to produce an antibody) in the host organism. Specifically, an antigen is a molecule that is bound by a binding site on an antibody. Typically, antigens are bound by antibody ligands and are capable of raising an antibody response in vivo. An antigen can comprise a polypeptide, protein, nucleic acid, lipid, and/or other molecule. The term antigen as used herein includes an epitope or antigenic determinant.

The term “epitope” or “antigenic determinant” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. The epitope can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

The term “aptamer” as used herein refers to single-stranded nucleic acids (e.g., DNA or RNA) that are approximately 20-100 bases in length. Aptamers generally spontaneously fold into 3-dimensional structures and can bind to specific target molecules (e.g., proteins, phospholipids, sugars, and other nucleic acids) with high specificity and affinity. Aptamers can generally identified through systemic evolution of ligands by exponential enrichment (SELEX). Similar to phage display library techniques, the aptamers that can bind to the target molecule more tightly are preferentially amplified by each round of selection. Aptamers are usually more resistant to pH and temperature changes than antibodies, and like peptides they can be easily synthesized and modified through chemical methods with low cost and less batch variance.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “specificity” refers to the number of different types of antigens or antigenic determinants to which a particular antibody or antigen-binding fragment thereof can bind. The specificity of an antibody or antigen-binding fragment or portion thereof, alone or in the context of a bispecific or multispecific polypeptide agent, can be determined based on affinity and/or avidity. The affinity, represented by the equilibrium constant for the dissociation (K_{D}) of an antigen with an antigen-binding protein (such as a bispecific or multispecific polypeptide agent), is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (KA), which is 1/K_{D}). As will be clear to the skilled person, affinity can be determined in a manner known per se, depending on the specific antigen of interest. Accordingly, a bispecific or multispecific polypeptide agent as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10,000 times or more better than the affinity with which said amino acid sequence or polypeptide binds to another target or polypeptide. Preferably, when a bispecific or multispecific polypeptide agent is “specific for” a target or antigen compared to another target or antigen, it is directed against said target or antigen, but not directed against such other target or antigen. Avidity is the measure of the strength of binding between an antigen-binding molecule (such as a bispecific polypeptide agent described herein) and the pertinent antigen. Avidity is related to both the affinity between an antigenic determinant and its antigen binding site on the antigen-binding molecule, and the number of pertinent binding sites present on the antigen-binding molecule. Specific binding of an antigen-binding protein to an antigen or antigenic determinant can be determined in any suitable manner known per se, including, for example, Scatchard analysis and/or competitive binding assays, such as radioimmunoassays (MA), enzyme immunoassays (EIA) and sandwich competition assays, and the different variants thereof known per se in the art; as well as other techniques as mentioned herein.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Compositions of the Disclosure

Compositions according to the disclosure are synthetic particle antibodies which comprise a bi-functional particle framework, such as for example and not limitation, a Janus micro- or nanoparticle, wherein one side of the bi-functional particle comprises targeting ligands and the other side of the bi-functional particle comprises immune-activating ligands (such as for example and not limitation, fragments of the Fc portion of antibodies, immune-activating peptides, and other proteins or peptides that mimic the structure and/or function of the Fc portion of antibodies). In one embodiment of the disclosure, the immune-activating ligands can directly or indirectly stimulate the subject's immune system. In another embodiment of the disclosure, the synthetic particle antibodies can be useful in diagnostic applications (non-limiting examples include synthetic particle antibodies with ligands that can be recognized by secondary fluorescent or radio-labeled antibodies, ligands that are conjugated to a radiotracer or a contrast agent, and/or ligands that are themselves contrast agents (non-limiting examples include gadolinium chelates, and/or radiotracers for contrast imaging such as CAT imaging, MRI imaging, PET imaging, and SPECT imaging). In another embodiment of the disclosure, the synthetic particle antibodies can be useful in research applications (non-limiting examples include ligands that can be recognized by secondary fluorescent or radio-labeled antibodies (e.g., antibodies that are useful in immunohistochemistry), ligands that can be utilized in immunoprecipitation (e.g., pull-down assays, column-based purification), and/or ligands that can be utilized in immunoblotting (e.g., Western blotting and enzyme-linked immunosorbent assays (ELISAs)).

### Targeting Ligands

The term “targeting ligands” as used herein includes, for example and not limitation, proteins, peptides, aptamers, lipids, carbohydrates, unnatural biomolecules, and/or fragments thereof that have the ability to specifically bind to a desired macromolecule (e.g., protein, peptide, lipid, carbohydrate, polysaccharide, and/or nucleic acid), cell or tissue type in a subject's body. The specific binding enables compositions of the disclosure to be directed or targeted to those cells or tissues of interest. The disclosure contemplates targeting ligands that are currently known, such as for example and not limitation, cancer specific targets (e.g., CD33, HER2 for breast cancers, CD52, CD20, EGFR), integrin α-4 on T-cells for multiple sclerosis, auto-antigens for autoimmune diseases, etc., as well as targeting ligands that have yet to be discovered.

Targeting ligands that can be used in compositions of the disclosure include, for example and not limitation, antigens and/or fragments thereof, epitopes and/or fragments thereof, protein fragments comprising a Fab fragment of an antibody, peptides, aptamers, lipids, polysaccharides, carbohydrates, unnatural biomolecules, and fragments of ligands that exist in the body for specific receptors on the cell targets. A molecule that bears high specificity and affinity to a cell or tissue target can also be a targeting ligand. One or more terminal amino acids of any peptide or protein targeting ligand or one or more terminal groups of any targeting ligands as described herein can be functionalized with different chemical groups for modification of the particle antibody.

In addition to known targeting ligands (e.g., peptides, proteins, Fab fragments, epitopes, aptamers, antigens, lipids, carbohydrates, unnatural biomolecules, and/or fragments thereof), new targeting ligands can be identified by, for example and not limitation, ScFv phage display libraries. After identification, vectors comprising the gene sequence encoding these peptides or proteins can be carefully designed in order to allow for chemical modification of the peptides or proteins for conjugating onto particle surfaces as described in more detail herein. Other methods of identifying new targeting ligands include phage display assays such as biopanning, as described in Ellis et al 2012 and Molek et al 2011, and aptamer discovery methods as described in Zhou J. 2017, and in Wang, A. Z. 2014

Non-limiting exemplary targeting ligands according to the disclosure are shown in the table below.

| Ligand | Sequence/ | | |
|---|---|---|---|
| type | Trade name | Target | Reference/Company |
| Peptide | WGWSLSHGYQ | Myeloid- | Hong, et al., Nature |
| | VK | derived | Medicine (2014), |
| | | suppressor | 20(6):676-681 |
| | | cells | |
| Peptide | FCGDGFYACY | P185HER2/ | Byeong-Woo Park et |
| | MDV | neutyrosine | al., Nature |
| | | kinases | Nanotechnology |
| | | | (2000), 18:194-198 |
| Peptide | LSLITRL | IL-6Ra | Su, J. L. et al., |
| | | | Cancer Res. 2005, |
| | | | 65, 4827-4835 |
| Peptide | YEQDPWGVKW | Tumor- | Maryelise Cieslewicz |
| | WY | associated | et al., PNAS (2013), |
| | | macrophages | 110 (40): 15919- |
| | | | 15924 |
| Aptamer | E-10030 | PDGF | Ophthotech/Retinal |
| | | | Consultants of |
| | | | Arizona |
| Aptamer | Macugen | VEGF1 | Pfizer/Eyetech |
| Antibody | Cetuximab | EGFR | N/A |
| Fragments | Fab | | |

### Immune-Activating Ligands

The term “immune-activating ligands” as used herein includes, for example and not limitation, proteins, peptides, fragments of the Fc portion of antibodies, immune-activating peptides, and other proteins or peptides that mimic the structure and/or function of the Fc portion of antibodies and/or fragments thereof that have the ability to activate or stimulate an immune response in a subject's body. The disclosure contemplates immune-activating ligands that are currently known, such as for example and not limitation, Pep33, or a Fc fragment from human IgG₁, as well as immune-activating ligands that have yet to be discovered, including but not limited to nucleic acids, lipids, carbohydrates, and unnatural biomolecules.

Immune-activating ligands that can be used in compositions of the disclosure include, for example and not limitation, fragments of the Fc portion of antibodies, immune-activating peptides, and other proteins or peptides that mimic the structure and/or function of the Fc portion of antibodies. In one embodiment, the immune-activating peptide comprises Pep33 (Bonetto et al 2009), which was identified through phage display library assays against human FcrRI and was shown to be capable of inducing phagocytosis activity and super-oxide burst of macrophages. Pep33 can thus be used as an Fc-mimicking peptide in synthetic particle antibodies of the disclosure as it can elicit anti-target immune responses. Other exemplary methods of identifying immune-activating ligands include aptamer screening and/or structural mimicking engineering.

When selecting Fc fragments for use in synthetic particle antibodies of the disclosure, the isotype and subclasses of the antibody should be considered based on the type of immune response that is desired. For example, if an allergic-type immune reaction is desired, an Fc fragment from an IgE antibody. If a complement activation is desired, an Fc fragment from an IgG3 is preferred, while IgG1 works better for soluble protein antigens or cells and IgG2 works better for bacterial capsular polysaccharide antigens.

### Particles

Particles that can be used in synthetic particle antibody compositions of the disclosure are bi-functional, meaning that they have surfaces with two or more distinct physical properties. These different physical properties enable two different types of chemistry to occur on the same particle. A non-limiting example of a particle according to the disclosure is a Janus particle.

The particles can be comprised of inorganic and/or organic materials and combinations thereof, such as for example and not limitation, metals, polymers, and/or lipids.

Commonly used metal particles include, for example and not limitation, gold (Au), silver (Ag), iron oxide, manganese, dysprosium, and holmium particles. Metal particles have been intensively researched as solid carriers of drugs. They have benefits such as enhancing drug bio-distribution to specific malignancies; protecting therapeutic molecules from detrimental effects; reducing non-specific interactions at non-targeted sites; and facilitating imaging and monitoring of the treatment efficacy as contrast agents. Metallic particles can also easily be fabricated into different sizes and/or shapes to alter the bio-distribution and pharmacokinetics. The long-term stability of metallic particles is also usually better than polymeric particles and lipid particles in liquid solutions. Similarly, iron oxide particles can be used for Mill imaging and are easily controlled by magnetic fields in manufacturing procedures.

Polymeric particles, such as for example and not limitation, polylactic acid (PLA), polyglycolic acid (PGA), polylactic-co-glycolic acid (PLGA), polyethyleneimine (PEI), chitosan, agarose, and polyethylene glycol (PEG), including polymersomes, have the advantages of being readily tunable by chemically modification of the constituent blocks of polymers to alter the drug loading efficiency, release kinetics, pharmacodynamics and targeting properties. Unlike metallic particles, polymer particles can both conjugate/load drugs/ligands on the surface and encapsulate biologics (e.g., proteins, antibodies, biological drugs/therapeutics) in the core, which facilitates the delivery of therapeutics, such as for example and not limitation, hydrophobic drugs that do not dissolve in aqueous solution. Thus, in embodiments comprising polymeric particles, the disclosure includes polymeric particles that encapsulate therapeutics within the particle itself, and/or polymeric particles that have drugs conjugated or loaded on the surface of the particle.

Lipid particles according to the disclosure are also liposomes. Because of their similarity to cells, the immunogenicity of the liposomes themselves is often much less than other particles. Like polymeric particles, liposomes can encapsulate drugs; drugs can also be loaded onto the surface of the liposomes. In addition, their size and surface chemistry can also be controlled for specific applications. As lipids possess high fluidity, it is not recommended to use lipid material alone for Janus particle fabrication. Lipids can be used to coat the surface of Janus particles or to form one hemisphere of Janus particle with other materials such as polymers (see, e.g., Garbuzenko et al). Thus, in embodiments comprising lipid particles, the disclosure includes lipid particles that encapsulate therapeutics within the particle itself, and/or lipid particles that have drugs conjugated or loaded on the surface of the particle.

The bi-functional particles of the invention can be of a variety of sizes and shapes, depending on the desired application. Size and shape should be determined according to the target tissues or the distribution of cell targets, while taking into consideration the renal clearance threshold (<10 nm to 15 nm) and interstitial/lymphatic fenestration (<20 nm) (see, e.g., Choi et al 2011, Shilo et al 2012, and Moghimi et al 2005). In general, about 20 nm-about 5000 nm is a preferred size range for synthetic particle antibodies of the disclosure as this not only provides enough surface area and volume for multivalent ligand presentation but also allows targeting of different organs/tissues via tuning size.

It is known that nanoparticles typically mostly go to liver and spleen, followed by lung, kidney, testis, thymus, heart and brain after intravenous injection. The decrease in nanoparticle size has been shown to lead to a decrease in distribution in the liver and spleen (see, e.g., Dreaden et al 2012). Smaller sized particles (nanometer range <10 nm) usually traverse most tissues freely; however, they generally diffuse away rapidly and get cleared faster into the subject's circulation. Larger particles tend to have less penetration into tissues but better retention in the tissues. Leaky vasculatures, such as those found in tumors, allow particles in the size range of about 20 nm to about 200 nm to extravasate. Nanoparticles approximately 30 nm to 100 nm in size have both good penetration and long retention; therefore, nanoparticles of about 30 nm to about 100 nm have been reported to be the optimal size range for anti-tumor drugs (see, e.g., Tang et al. 2014; Matsumoto et al. 2016; Cabral et al. 2015). To target malignancies in the lymph node, smaller particles, such as about 20 nm to about 40 nm, can be used. It is known that smaller nanoparticles generally have a longer half-life in blood. Thus, the use of smaller particles (20 nm to about 40 nm) may be suggested for detecting targets in blood.

The size and shape of particles also dictates different optical properties of particles, especially metallic particles. For example, the larger the gold spheres, the higher the surface plasmon resonance (SPR) peak wavelength is. Gold nanospheres usually have an SPR peak at much lower than 1000 nm, while gold nanorods can have a peak at IR range (1000-1100 nm) by controlling the length and diameter of the rods. These properties can be employed for imaging when a synthetic particle antibody of the disclosure is used as contrast agent or for phototherapy when a synthetic particle antibody of the disclosure is composed of gold particles.

Micron-sized particles are more prone to be phagocytosed by phagocytes during circulation before they reach their target tissue. Generally, a synthetic particle antibody of the disclosure is intended to opsonize the target cells and activate the Fc receptors on an immune cell surface, and thus are preferentially nanoparticles. Micron-sized particles can be used in certain embodiments of the disclosure, such as for example and not limitation about 1 um to about 2 um.

a. When a larger volume of the synthetic particle antibody is needed, such as for modification with ligands to achieve the desired functional outcome, or for encapsulation of a drug or biologic;

b. As micro-sized particles possess higher avidity and similarity to a cell, it may trigger an enhanced immune activation. So, if the synthetic particle antibody surface is carefully designed (e.g. PEGylated) to avoid phagocytosis, or if synthetic particle antibodies are injected via routes other than intravenous (e.g., subcutaneous), large micron-sized particles can be considered;

c. Microparticles have been shown to facilitate better cross-presentation and elicit T cell immune response. Therefore, micro-sized synthetic particle antibodies could be helpful when the formation of an immune memory is desired;

d. In diagnosis and/or research settings, where synthetic particle antibodies are used for immunoprecipitation or detection of targets ex vivo, micron-sized particles have advantages of higher valency and thus potentially increase the specificity and lower the detection limits of the particles.

Besides size and surface chemistry, the shape also affects cellular uptake of the particles (see, e.g., Dreaden et al 2012 and Tang, L. et al 2014). Shapes with a higher length-width ratio generally result in lower uptake. Therefore, rods and/or discs can be considered when the cell target is highly phagocytic or low in numbers such that a longer half-life of the synthetic particle antibody is needed.

Bi-functional particles can be made by a variety of different methods now known or later developed. One exemplary type of method is a solid phase chemistry method (see, e.g., Peiris P M et al 2011). The solid phase chemistry methods generally enable the fabrication of Janus particles with a solid surface via a cleavable crosslinker. Cleavage results in new functional groups added onto a portion of the particles' surface that interacted with the solid surface. The new functional groups define the bioconjugation chemistry to use for additional modification(s) of the surface with ligands. This method has the advantage of simple set-up and process as well as bulk production of large quantities of Janus particles in one shot.

Another exemplary type of method is a droplet microfluidics method (see, e.g., Saifullah et al 2014 and Zhihong N et al 2006). This method generally includes the set-up of a microfluidic device and preparation different chemical and/or biological substance solutions. Three major flow regimes (co-flow, double emulsion, and phase separation) can be employed to fabricate Janus particles. Merging of two different monomers/polymeric/ceramic/metallic materials from separate channels in the presence of an electric/magnetic field or photo-initiator and UV light (for example and not limitation) can lead to the formation of Janus particles in various nano-scale or micro-scale ranges. Different choices of materials provide different options for conjugation of ligands onto these Janus particles. The microfluidics method is competitive in terms of one-single step process and fast speed of production.

Other exemplary methods (see, e.g., Saifullah et al 2014, Jing H et al 2012, and Tang J et al 2012) include modifying a uniform, closely packed layer of particles with a metal coating (e.g., gold) on one hemisphere. Ligands can be attached in various ways (e.g., passive adsorption, chemical linking) to the active, spatial-segregated surfaces. Other methods include pickering emulsion, deposition of evaporated metal particles, layer-by-layer self-assembly and so on. Careful choice of the material would dictate how the modification with ligands would carry out.

### Methods of Making Synthetic Particle Antibodies of the Disclosure

A variety of chemical methods can be used to conjugate different ligands to the surface of the particle to generate synthetic particle antibodies according to the disclosure.

An exemplary conjugation method involves amine reactions. There is a list of NHS-esters available to react with amine groups either on the ligands or the particles. Amination is the simplest, most common reaction to label/crosslink peptides and proteins and typically occurs on the primary amines existing at the N-terminus of each peptide chain and/or in the side-chain of lysine amino acid residues where accessible in the protein or modified oligonucleotides at physiological pH. Other chemical groups that can form chemical bounds with amine groups include isothiocyanates, isocyanates, acyl azides, sulfonyl chlorides, aldehydes, epoxides, carbodiimides, anhydrides. etc.

Another exemplary conjugation method involves a sulfhydryl-maleimide reaction. This reaction is another class of reaction that can be utilized to specifically conjugate ligands on the surface of particles. Sulfhydryl groups usually exist in the side chain of cysteines, or can be created by breakage of disulfide bonds in the protein/ligands, provided that native structure and functions of the protein/ligands will not be affected by the cleavage. Sulfhydryl-reactive chemical groups include haloacetyls, maleimide, arylating agents, vinylsulfones, TNB-thiols and disulfide reducing agents. Most of these groups conjugate to sulfhydryls by either alkylation or disulfide exchange.

Another exemplary conjugation method involves a biotin-streptavidin/avidin/NeutrAvidin reaction. The advantages of this reaction are its high specificity and wide working conditions (e.g., temperature, pH). Biotinylation reagents are readily available for various functional groups existing in the biological molecules, such as primary amines, sulfhydryls, carboxyls and carbohydrates.

Other exemplary conjugation methods involve:

a) Staudinger reagent pairs: Staudinger ligation reagents are pairs of metabolic or chemical labeling compounds that have azide and phosphine groups, respectively. These groups do not naturally exist in the biomolecules, so the ligands and particles have to be labeled with these chemical groups to link to each other.

b) Click-chemistry: For example, Cu(I)-catalyzed and copper-free azide-alkyne cycloaddition has already been intensively applied in the functionalization of particle-delivery system of drug. This chemistry is simple and high yielding and thus promising as a bioconjugation tool for particle surface modification.

c) Photo-initiated reactions: There are a handful of photo-chemical reactive groups available for bioconjugation. The most widely used is aryl azides, while psoralen almost exclusively reacts with nucleo-acids, in which case can be applied for aptamers as ligands.

d) PEGylation: PEGylation is suggested to reduce the opsonization of the particles, increase blood circulation time and modify the surface morphology of the synthetic particle antibodies as needed.

### Therapeutic Applications of Compositions of the Disclosure

In some embodiments, the synthetic particle antibodies of the disclosure are used to treat and/or prevent certain diseases and/or conditions, such as for example and not limitation, cancers, tumors, autoimmune diseases, and/or various infections.

Non-limiting examples of cancers treatable by the compositions and methods of the disclosure include, for example, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstrom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies of all types including but not limited to EBV-associated Hodgkin's and non-Hodgkin's lymphoma, all forms of post-transplant lymphomas including post-transplant lymphoproliferative disorder (PTLD), uterine cancer, renal cell carcinoma, hepatoma, hepatoblastoma, etc.

Non-limiting examples of the inflammatory and autoimmune diseases treatable by the compositions and methods of the present disclosure include, e.g., inflammatory bowel disease (IBD), graft-versus host disease (GVHD), ulcerative colitis (UC), Crohn's disease, diabetes (e.g., diabetes mellitus type 1), multiple sclerosis, arthritis (e.g., rheumatoid arthritis), Graves' disease, lupus erythematosus including systemic lupus erythematosus, ankylosing spondylitis, psoriasis, Behcet's disease, autistic enterocolitis, Guillain-Barre Syndrome, myasthenia gravis, pemphigus vulgaris, acute disseminated encephalomyelitis (ADEM), transverse myelitis autoimmune cardiomyopathy, Celiac disease, dermatomyositis, Wegener's granulomatosis, allergy, asthma, contact dermatitis, atherosclerosis (or any other inflammatory condition affecting the heart or vascular system), autoimmune uveitis, as well as other autoimmune skin conditions, autoimmune kidney, lung, or liver conditions, autoimmune neuropathies, etc. In some embodiments, the composition or method of the disclosure is used to treat systemic lupus erythematosus, multiple sclerosis, and GVHD.

In a related embodiment, the compositions and methods of the disclosure can be used to treat or prevent tissue or organ rejection in a recipient receiving a transplant. For example and not limitation, the compositions and methods of the disclosure can be used to prevent rejection of transplanted kidney tissue (or organ) or liver tissue (or organ) in a transplant recipient.

It is contemplated that when used to treat various diseases, the compositions and methods of the present disclosure can be combined with other therapeutic agents suitable for the same or similar diseases. Also, two or more embodiments of the disclosure may be also co-administered to generate additive or synergistic effects. When co-administered with a second therapeutic agent, the embodiment of the disclosure and the second therapeutic agent may be simultaneously or sequentially (in any order). Suitable therapeutically effective dosages for each agent may be lowered due to the additive action or synergy.

As a non-limiting example, the disclosure can be combined with other therapies that block inflammation (e.g., via blockage of ILL IFNα/β, IL6, TNF, IL13, IL23, etc.).

The compositions and methods of the disclosure can be also administered in combination with an anti-tumor antibody or an antibody directed at a pathogenic antigen or allergen.

The compositions and methods of the disclosure can be combined with other immunomodulatory treatments such as, e.g., therapeutic vaccines (including but not limited to GVAX, DC-based vaccines, vaccines against specific cancer antigens, etc.), checkpoint inhibitors (including but not limited to agents that block CTLA4, PD1, LAG3, TIM3, etc.) or activators (including but not limited to agents that enhance 41BB, OX40, etc.). The inhibitory treatments of the disclosure can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e), in any of the aforementioned forms or formulations, alone or in combination with each other or other agents.

Therapeutic methods of the disclosure can be combined with additional immunotherapies and therapies. For example, when used for treating cancer, the synthetic particle antibodies of the disclosure can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors of the disclosure include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the synthetic particle antibody compositions of the disclosure can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, BCG, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

For treatment of infections, a combined therapy of the disclosure can encompass co-administering compositions and methods of the disclosure with an antibiotic, an anti-fungal drug, an anti-viral drug, an anti-parasitic drug, an anti-protozoal drug, or a combination thereof.

Non-limiting examples of useful antibiotics include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); vancomycins; bacitracins; macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Pefloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. See also Physician's Desk Reference, 59th edition, (2005), Thomson P D R, Montvale N.J.; Gennaro et al., Eds. Remington's The Science and Practice of Pharmacy, 20th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J. Such antibiotics can be obtained commercially, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Non-limiting examples of useful anti-fungal agents include imidazoles (such as griseofulvin, miconazole, terbinafine, fluconazole, ketoconazole, voriconazole, and itraconizole); polyenes (such as amphotericin B and nystatin); Flucytosines; and candicidin or any salts or variants thereof. See also Physician's Desk Reference, 59th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

Non-limiting examples of useful anti-viral drugs include interferon alpha, beta or gamma, didanosine, lamivudine, zanamavir, lopanivir, nelfinavir, efavirenz, indinavir, valacyclovir, zidovudine, amantadine, rimantidine, ribavirin, ganciclovir, foscarnet, and acyclovir or any salts or variants thereof. See also Physician's Desk Reference, 59th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

Non-limiting examples of useful anti-parasitic agents include chloroquine, mefloquine, quinine, primaquine, atovaquone, sulfasoxine, and pyrimethamine or any salts or variants thereof. See also Physician's Desk Reference, 59^{th} edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.

Non-limiting examples of useful anti-protozoal drugs include metronidazole, diloxanide, iodoquinol, trimethoprim, sufamethoxazole, pentamidine, clindamycin, primaquine, pyrimethamine, and sulfadiazine or any salts or variants thereof. See also Physician's Desk Reference, 59^{th} edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.

In some embodiments, the synthetic particle antibodies of the disclosure can be used to specifically target and/or deplete immune suppressor cells and/or cancer cells, thus treating and/or preventing cancer. In such embodiments, the synthetic particle antibodies are engineered with targeting ligands against immune suppressor cells and/or cancer cells. In some embodiments, the immune-activating ligands on the opposite face of the synthetic particle antibody can bind to Fc receptors on immune cells and facilitate antibody-dependent cell killing, such as for example and not limitation, use of Pep33 peptides to target and deplete myeloid-derived suppressor cells as shown in more detail herein. Other targeting ligands contemplated by the disclosure can target the synthetic particle antibodies to other immune suppressor cells (e.g., regulatory T-cells), to well-studied and validated cancer specific targets (e.g. CD33, HER2, CD52, CD20, EGFR), and/or to novel disease-specific targets that can be identified using phage display or other methods as discussed herein.

In further embodiments, the synthetic particle antibodies of the disclosure that are adapted for treating and/or preventing cancer, the synthetic particle antibodies can be used in combination with other cancer therapies as discussed herein, such as for example and not limitation, with cancer vaccines, chemotherapeutics, and radiation-based chemotherapy. Without wishing to be bound by theory, it is suggested that cancer vaccine efficacy can often be limited by the presence of checkpoint blockade and immune suppressor cells, which can thus limit the extent of the immune response in the tumor. The synthetic particle antibodies of the disclosure could be used to deplete myeloid-derived suppressor cells (MDSCs) and/or tumor-associated macrophages (TAMs), thereby possibly removing one mechanism of immune suppression and subsequently enhancing the immunogenicity of the cancer vaccine. In some embodiments, the synthetic particle antibodies of the disclosure (e.g., polymer-based particles) could be designed to encapsulate chemotherapeutics within the particle core, and/or the synthetic particle antibodies could be delivered in combination with existing chemotherapy regimens. One challenge with chemotherapy is the existence of intracellular resistance mechanisms that hinder therapeutic efficacy. The synthetic particle antibodies of the disclosure can enable killing by two mechanisms (antibody-mediated and chemotherapy mediated), which can enhance overall therapeutic efficacy. The synthetic particle antibodies of the disclosure can be delivered in conjunction with radiation therapy for cancer patients. While radiation therapy is successful at inducing apoptosis in tumors, it also forms an environment that is favorable for the proliferation of regulatory T cells that can negate the anti-tumor effect. Gold particle-based synthetic particle antibodies can be used to deplete immune-suppressor cells (e.g., MDSCs) and at the same time assist phototherapy for cancer destruction, as a result of which a immune-promoting environment is created for T cell to eliminate tumor cells. Targeted depletion of regulatory T cells could enable improved outcomes with radiation therapy. Alternatively, synthetic particle antibodies of the disclosure could be engineered to target tumor cells that have upregulated ligands facilitating checkpoint blockade (e.g., PD-L1) to promote an anti-tumor effect.

In other embodiments, synthetic particle antibodies of the disclosure can be engineered with targeting ligands that recognize T cells in subjects with autoimmune diseases, such as for example and not limitation, systemic lupus erythematosus (SLE), and can thus treat and/or prevent the autoimmune disease by depleting such T cells. In other embodiments, synthetic particle antibodies of the disclosure can be engineered with targeting ligands to specifically detect idiotypes on autoantibodies to deplete B cells that recognize the same auto-antigen, and thus can also be used to treat and/or prevent the autoimmune disease by depleting such B cells. In still other embodiments, specifically for treating and/or preventing multiple sclerosis (MS), synthetic particle antibodies of the disclosure can be engineered with targeting ligands to recognize specific integrins on the surface of T cells, which can prevent T cell proliferation into central nervous system (CNS) lesions. Other MS-specific therapies that are contemplated by the disclosure include the use of synthetic particle antibodies of the disclosure can be engineered with targeting ligands to specifically detect and deplete monocytes and lymphocytes in the bloodstream, and in further embodiments can be used to treat and/or prevent relapsing-remitting MS.

In other embodiments, synthetic particle antibodies of the disclosure can be engineered with targeting ligands that specifically recognize viruses, bacteria, parasites, fungi, and other disease-causing microorganisms.

In other embodiments, synthetic particle antibodies of the disclosure can be engineered with targeting ligands that specifically recognize and bind to the IL-2 receptor on T-cells, which could prevent T-cell activation and subsequent B-cell activation in kidney transplant recipients.

In other embodiments, synthetic particle antibodies of the disclosure can be engineered with targeting ligands that specifically recognize TNF-alpha, IL-12, or IL-23, all of which are cytokines that lead to severe inflammation in inflammatory bowel disease (IBD).

In other embodiments, synthetic particle antibodies of the disclosure can be engineered with targeting ligands that specifically recognize IL-17a and TNF-alpha, both of which are cytokines that are implicated in psoriasis.

In other embodiments, synthetic particle antibodies of the disclosure can be engineered with targeting ligands that specifically recognize TNF-alpha and/or IL-4, both of which are cytokines that are implicated in GVHD.

In any of the above embodiments, the size and/or shape of the synthetic particle antibody can be modified based on the target tissue, organ, and/or disease or condition being treated and/or prevented as discussed in more detail herein.

### Administration of Therapeutic Compositions of the Disclosure

The compositions of the disclosure can comprise a carrier and/or excipient. While it is possible to use a compound of the present disclosure for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice.

In one embodiment of any of the compositions of the disclosure, the composition is formulated for delivery by a route such as, e.g., oral, topical, rectal, mucosal, sublingual, nasal, naso/oro-gastric gavage, parenteral, intraperitoneal, intradermal, intramuscular, transdermal, intratumoral, intrathecal, nasal, and intratracheal administration. In one embodiment, the composition is formulated for delivery by a route such as, e.g., oral, nasal, intravascular, intraperitoneal, intratumoral, and transdermal administration. In one embodiment of any of the compositions of the disclosure, the composition is in a form of a liquid, foam, cream, spray, powder, or gel. In one embodiment of any of the compositions of the disclosure, the composition comprises a buffering agent.

Administration of the compounds and compositions in the methods of the disclosure can be accomplished by any method known in the art. Non-limiting examples of useful routes of delivery include oral, rectal, fecal (by enema), and via naso/oro-gastric gavage, as well as parenteral, intraperitoneal, intradermal, intramuscular, transdermal, intratumoral, intrathecal, nasal, and intratracheal administration. The active agent may be systemic after administration or may be localized by the use of regional administration, intratumoral administration, or use of an implant that acts to retain the active dose at the site of implantation.

The useful dosages of the compounds and formulations of the disclosure can vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses may be effective to achieve a therapeutic effect. While it is possible to use a compound of the present disclosure for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice. Although there are no physical limitations to delivery of the formulations of the present disclosure, intravenous delivery is preferred for delivery.

Oral delivery may also include the use of nanoparticles that can be targeted, e.g., to the GI tract of the subject, such as those described in Yun et al., Adv Drug Deliv Rev. 2013, 65(6):822-832 (e.g., mucoadhesive nanoparticles, negatively charged carboxylate- or sulfate-modified particles, etc.). Non-limiting examples of other methods of targeting delivery of compositions to the GI tract are discussed in U.S. Pat. Appl. Pub. No. 2013/0149339 and references cited therein (e.g., pH sensitive compositions [such as, e.g., enteric polymers which release their contents when the pH becomes alkaline after the enteric polymers pass through the stomach], compositions for delaying the release [e.g., compositions which use hydrogel as a shell or a material which coats the active substance with, e.g., in vivo degradable polymers, gradually hydrolyzable polymers, gradually water-soluble polymers, and/or enzyme degradable polymers], bioadhesive compositions which specifically adhere to the colonic mucosal membrane, compositions into which a protease inhibitor is incorporated, a carrier system being specifically decomposed by an enzyme present in the colon).

For oral administration, the active ingredient(s) can lyophilized along with a cryoprotectant and/or lyoprotectant, and can then be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

Solutions or suspensions can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity, such as sodium chloride or dextrose.

In instances in which the agents exhibit insufficient solubility, methods for solubilizing agents may be used. Such methods are known to those of skill in this art, and include, but are not limited to, using co-solvents, such as, e.g., dimethylsulfoxide (DMSO), using surfactants, such as TWEEN® 80, or dissolution in aqueous sodium bicarbonate. Pharmaceutically acceptable derivatives of the agents may also be used in formulating effective pharmaceutical compositions.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art (e.g., Remington's Pharmaceutical Sciences, Mack Publishing Company, Easton, Pa., 15th Edition, 1975). The composition or formulation to be administered will, in any event, contain a quantity of the active agent in an amount sufficient to alleviate the symptoms of the treated subject.

The active agents or pharmaceutically acceptable derivatives may be prepared with carriers that protect the agent against rapid elimination from the body, such as time release formulations or coatings. The compositions may include other active agents to obtain desired combinations of properties.

Oral pharmaceutical dosage forms include, by way of example and without limitation, solid, gel and liquid. Solid dosage forms include tablets, capsules, granules, and bulk powders. Oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

Parenteral administration, generally characterized by injection, either subcutaneously, intramuscularly or intravenously, is also contemplated herein. Injectables can be prepared in conventional forms, either as liquid solutions or suspensions, solid forms suitable for solution or suspension in liquid prior to injection, or as emulsions. Suitable excipients include, by way of example and without limitation, water, saline, dextrose, glycerol or ethanol. In addition, if desired, the pharmaceutical compositions to be administered may also contain minor amounts of non-toxic auxiliary substances, such as wetting or emulsifying agents, pH buffering agents, stabilizers, solubility enhancers, and other such agents, such as, for example, sodium acetate, sorbitan monolaurate, triethanolamine oleate and cyclodextrins.

Implantation of a slow-release or sustained-release system, such that a constant level of dosage is maintained (e.g., U.S. Pat. No. 3,710,795) is also contemplated herein. Briefly, an inhibitor of Nt5e or A1R is dispersed in a solid inner matrix (e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinylacetate) that is surrounded by an outer polymeric membrane (e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinylacetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer) that is insoluble in body fluids. The agent diffuses through the outer polymeric membrane in a release rate controlling step. The percentage of active agent contained in such parenteral compositions is highly dependent on the specific nature thereof, as well as the activity of the agent and the needs of the subject.

Lyophilized powders can be reconstituted for administration as solutions, emulsions, and other mixtures or formulated as solids or gels. The sterile, lyophilized powder is prepared by dissolving an agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution can be apportioned into vials for lyophilization. Each vial can contain, by way of example and without limitation, a single dosage or multiple dosages of the agent. The lyophilized powder can be stored under appropriate conditions, such as at about 4° C. to room temperature. Reconstitution of this lyophilized powder with water or other suitable carrier for injection provides a formulation for use in parenteral administration. The precise amount depends upon the selected agent. Such amount can be empirically determined.

The inventive composition or pharmaceutically acceptable derivatives thereof may be formulated as aerosols for application e.g., by inhalation or intranasally (e.g., as described in U.S. Pat. Nos. 4,044,126, 4,414,209, and 4,364,923). These formulations can be in the form of an aerosol or solution for a nebulizer, or as a microtine powder for insufflation, alone or in combination with an inert carrier such as lactose. In such a case, the particles of the formulation can, by way of example and without limitation, have diameters of less than about 50 microns, such as less than about 10 microns. For particles less than 1 um in size, a carrier (e.g., polymer microparticles) can be used to deliver the formulation to lungs for treatment of lung cancer or tuberculosis, or targeting of immune-suppressive cells in the lung for treatment of other diseases.

The agents may be also formulated for local or topical application, such as for application to the skin and mucous membranes (e.g., intranasally), in the form of nasal solutions, gels, creams, and lotions.

Other routes of administration, such as transdermal patches are also contemplated herein. Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

### Diagnostic Applications of Compositions of the Disclosure

In some embodiments, synthetic particle antibodies of the disclosure can be used in diagnostic applications, such as for example and not limitation, imaging (for both diagnosing a disease and for monitoring disease progression), and antibody-based diagnostics (such as for example and not limitation, enzyme-linked immunosorbent assays such as for example and not limitation, tests for determining the presence of HIV, Mycobacterium antibodies, rotavirus, hepatitis B, Lyme disease, Rocky Mountain spotted fever, squamous cell carcinoma, syphilis, toxoplasmosis, varicella-zoster virus, Zika virus, and enterotoxins in a subject's blood sample, as well as drug screening assays).

In such embodiments, synthetic particle antibodies of the disclosure can be formulated using a particle core that also serves as a contrast agent. The particle core can function as a contrast agent by, for example and not limitation, being a contrast agent itself (e.g., a metal or metal oxide particle), having a contrast agent encapsulated in the particle itself, and/or having the contrast agent functionally attached to the particle. The contrast agent enables the synthetic particle antibodies to detect cell targets determined by the targeting ligands on the opposite surface of the bi-functional particle. Non-limiting examples of particle contrast agents include iron oxide nanoparticles for MRI imaging or gold nanoparticles for x-ray computed tomography or photoacoustic imaging.

In any of the above embodiments, the size and/or shape of the synthetic particle antibody can be modified based on the specific diagnostic application as discussed in more detail herein.

### Research Applications of Compositions of the Disclosure

In some embodiments, the synthetic particle antibodies of the disclosure can be used in various research applications involving antibodies, such as for example and not limitation, immunoprecipitation, immunohistochemistry, and/or immunoblotting. It is intended that the synthetic antibodies of the disclosure can replace non-synthetic antibodies in these applications.

When used in immunoprecipitation applications such as for example and not limitation, pull-down assays and column-based purification, the synthetic particle antibodies of the disclosure can be engineered with targeting ligands on one face and immune-activating ligands on the opposite face that can be recognized by a bead (e.g., agarose, iron oxide, polypropylene gel), which allows the separation of antibody-antigen complexes by size and/or affinity to the receptor on the bead. Depending on the size of the core nanoparticle, the whole construct could also facilitate a one-step method to separate antibody-antigen complexes.

When used in immunohistochemistry applications such as for example and not limitation, antigen staining in a tissue of interest, the synthetic particle antibodies of the disclosure can be engineered with targeting ligands on one face and immune-activating ligands on the opposite face that can be recognized by a secondary fluorescent and/or radioactive antibody. The secondary antibody can enable the use of the synthetic particle antibodies for staining tissue for histological sections.

When used in immunoblotting applications such as for example and not limitation, Western blotting and enzyme-linked immunosorbent assays, the synthetic particle antibodies of the disclosure can be engineered with targeting ligands on one face and immune-activating ligands on the opposite face that can be recognized by a secondary fluorescent and/or radioactive antibody. The secondary antibody can enable the use of the synthetic particle antibodies for detecting binding of the synthetic particle antibody to a target of interest.

### Examples

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Example 1: Preparation of Synthetic Particle Antibodies

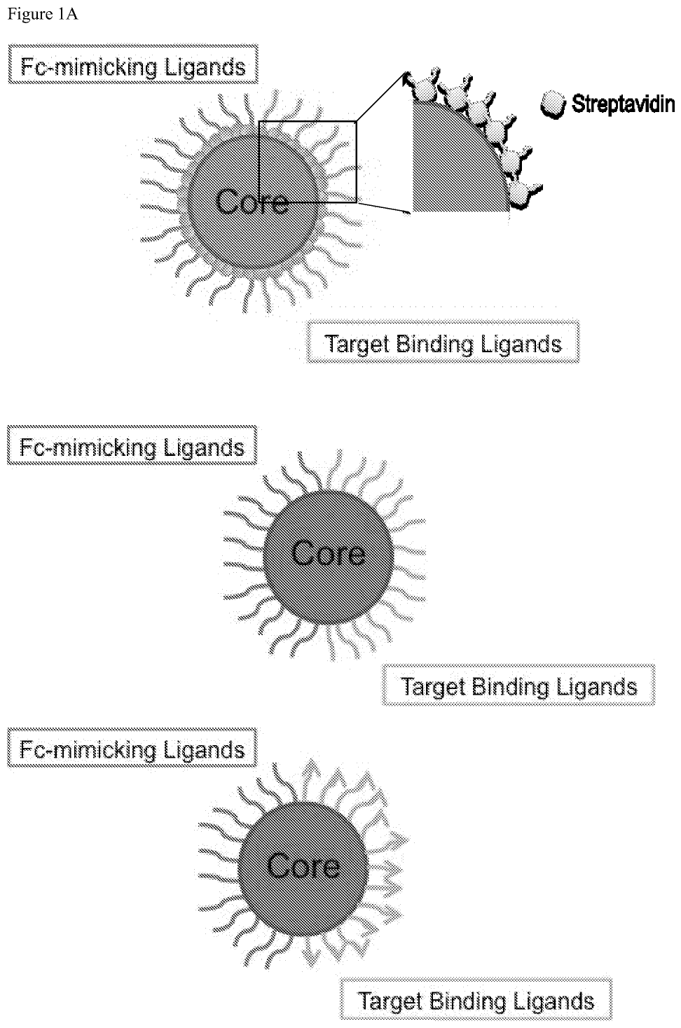

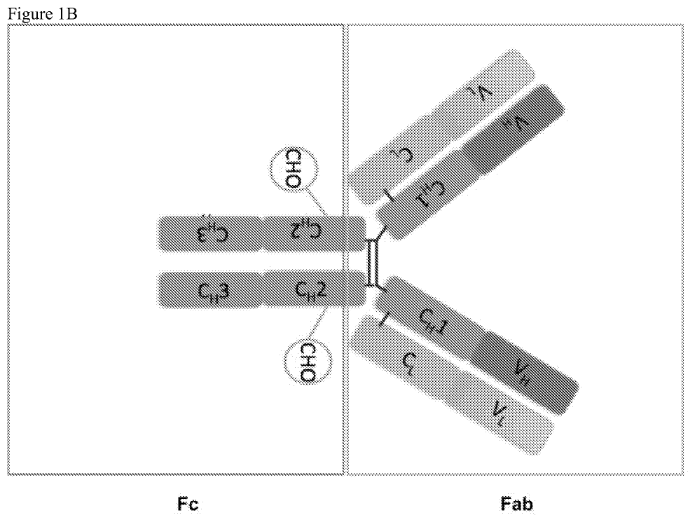

Three exemplary synthetic particle antibodies of the disclosure were depicted in FIG. 1A. The synthetic particle antibodies are generated by modifying the two hemispheres of bi-functional particles with targeting ligands on one side and immune-activating ligands on the other side through separate chemical reactions.

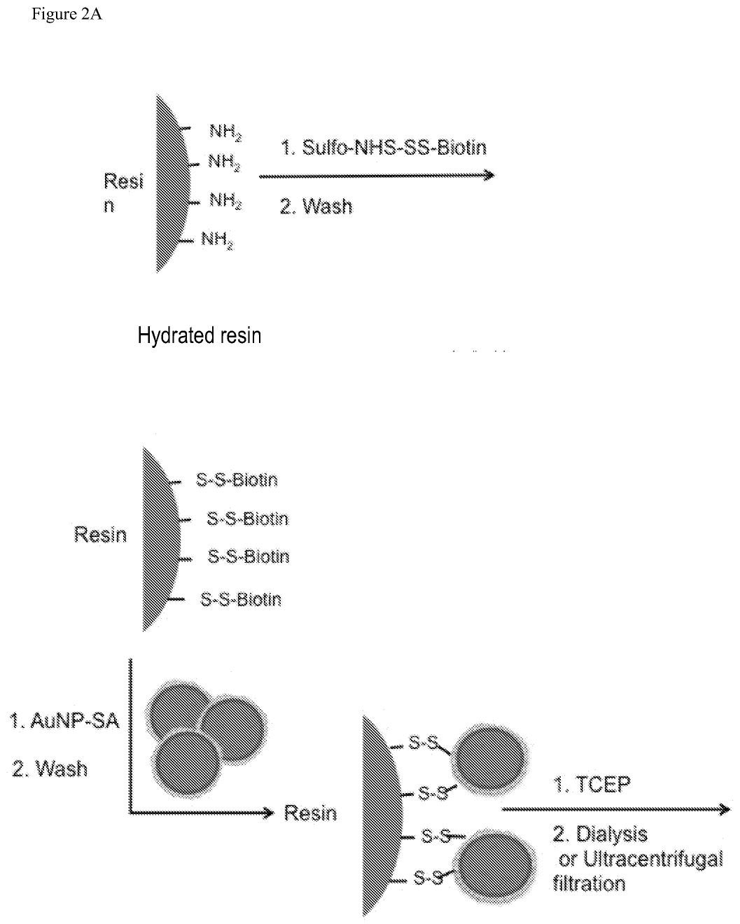

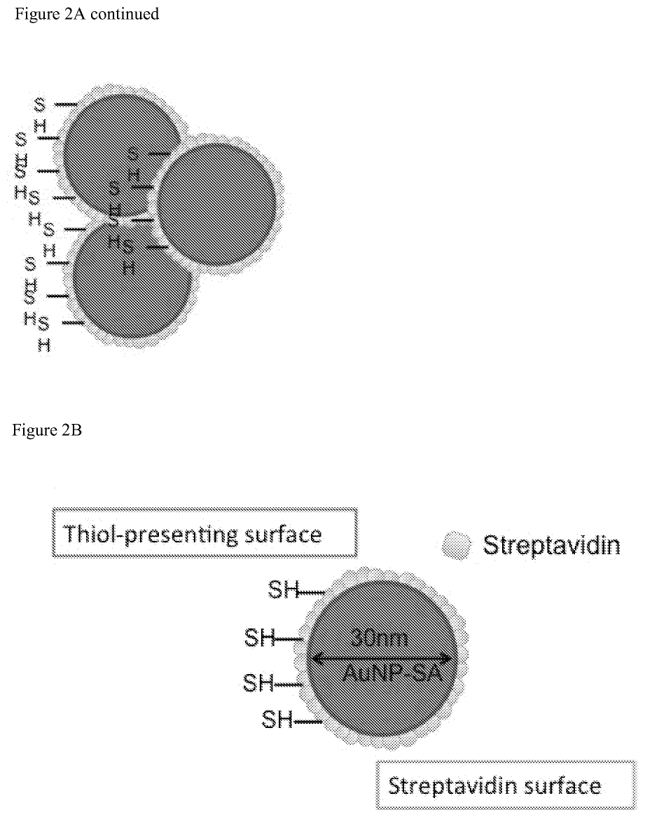

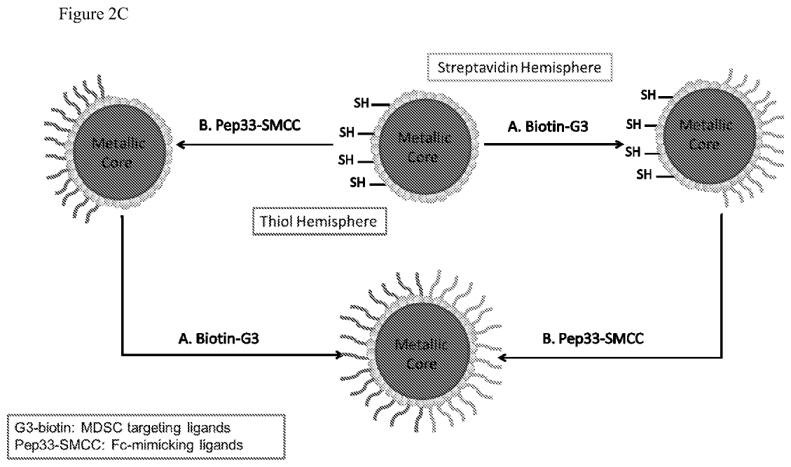

FIGS. 2A-2C depict one method of making synthetic particle antibodies according to the disclosure. Other methods of making bi-functional particles and conjugating targeting ligands and immune-activating ligands are specifically contemplated herein. The exemplary method shown in FIGS. 2A-2C is a synthetic particle antibody fabrication procedure using solid-phase chemistry. FIG. 2A showed a fabrication procedure of Janus gold nanoparticles from streptavidin-modified gold nanoparticles. Janus particles were generated by binding streptavidin-coated nanoparticles onto biotin-s-s-sulfo-NHS crosslinker-functionalized amine-presenting resins. Cleavage of the disulfide bounds led to the formation of thiol group displaying hemisphere on the Janus particles. (FIG. 2B) Structure of Janus gold nanoparticles. The Janus gold nanoparticles resulting from the solid phase chemistry in FIG. 2A had one hemisphere with thiol groups and the other hemisphere with free biotin-binding sites on streptavidin. (FIG. 2C) Modification of Janus gold nanoparticles with ligands (G3-Biotin and Pep33-SMCC as examples). Pep33 peptides, as an example of immune-activating ligands (and specifically Fc-mimicking ligands), were conjugated onto the thiol-presenting hemisphere via maleimide-SH reaction in physiological pH (pH 7.0-7.4) in PBS. G3 peptides, as an example of targeting ligands, were conjugated onto the free-biotin hemisphere through streptavidin-biotin interaction in PBS. After removal of excessive ligands, synthetic particle antibodies with targeting ligands and Fc-mimicking ligands on different hemisphere were generated.

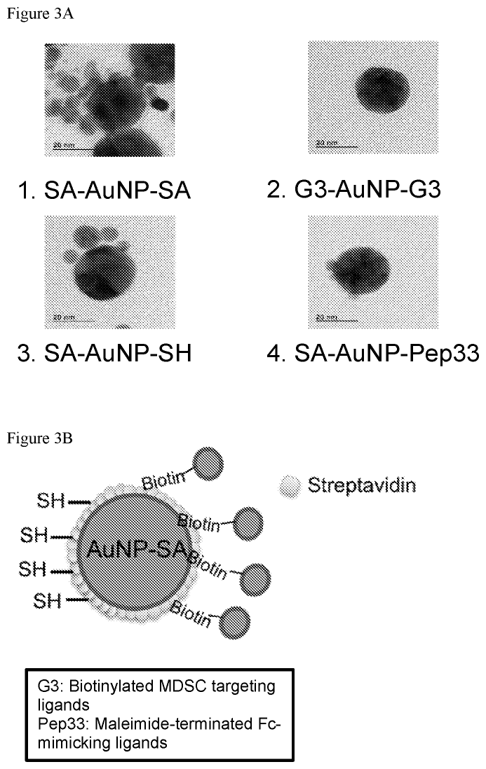

FIG. 3A provided validation of the Janus conformation of the nanoparticles produced from solid phase chemistry described in FIGS. 2A-2B. 3 nm biotin-gold nanoprobes were bound onto the free biotin-binding sites on the unmodified streptavidin coated gold nanoparticles. 3 nm biotin-gold nanoprobes could not bind after the entirety of the streptavidin-coated gold nanoparticles were modified with biotinylated targeting ligands (G3). On the contrary, multiple 3 nm biotin-gold nanoprobes could bind to the free-biotin binding sites on one face of the Janus gold nanoparticles before and after modification on the opposite face with maleimide-Fc-mimicking ligands (Pep33-SMCC). Taken together, the gold nanoparticles generated from the designed solid phase chemistry have a bi-functional or Janus surface structure. Moreover, the Janus particles have multiple binding sites, enabling the multivalent presentation of ligands. FIG. 3B provided a diagrammatic representation of such synthetic particle antibodies.

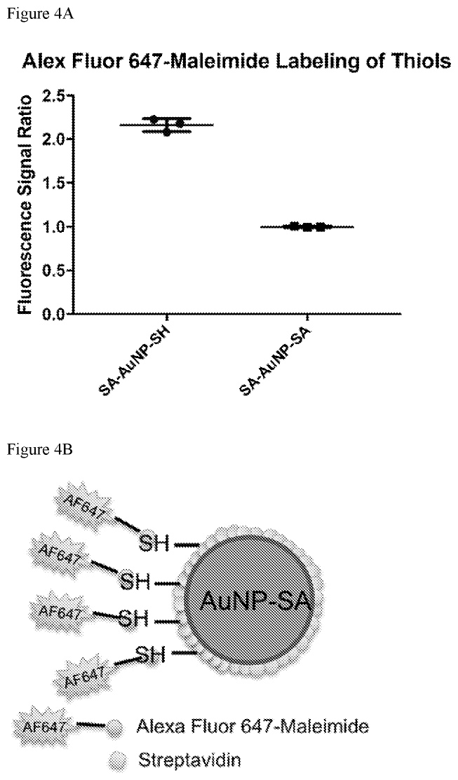

FIG. 4A demonstrated validation of the existence and availability of thiol groups for maleimide reactive groups (coupled to the immune-activating ligands). Janus nanoparticles generated from the solid phase chemistry method were reacted with Alexa Fluor 647-Maleimide dye (Thermo Fisher, InVitrogen™, Cat. No. A20347), followed by dialyzing against PBS to remove the unbound dye. The increase of the fluorescence signal in the Janus gold nanoparticle group (SA-AuNP-SH) compared to the unmodified gold nanoparticle group (SA-AuNP-SA) indicated the occurrence of the reaction between the maleimide group on the Alexa Fluor 647 dye and the thiol groups on the Janus particles, resulting in conjugation of the immune-activating ligands. FIG. 4B provided a diagrammatic representation of such synthetic particle antibodies.

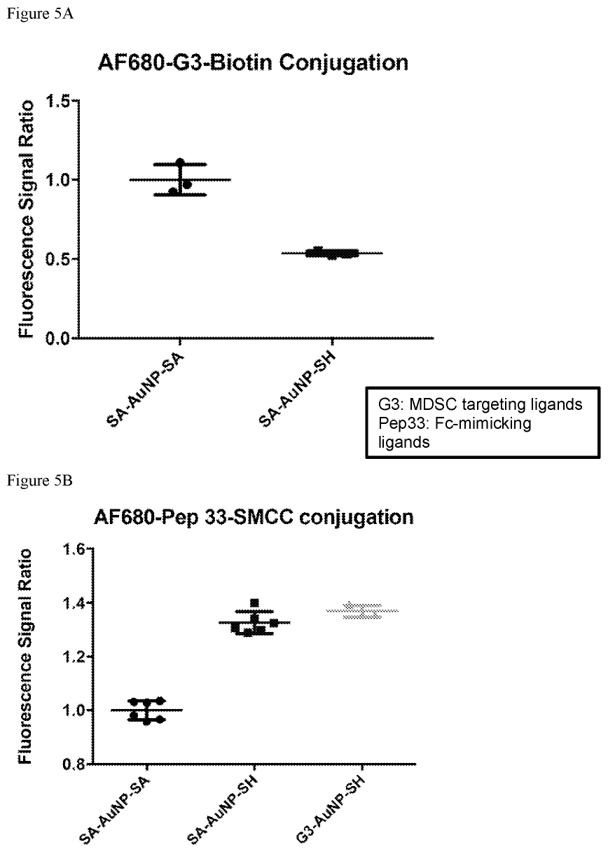

FIGS. 5A-5B provide validation of peptide modification on Janus gold nanoparticles with fluorescently labeled peptide ligands. As an example, targeting ligands G3-biotin (FIG. 5A) and Fc-mimicking ligands Pep33-SMCC (FIG. 5B) were both tagged with Alexa Fluor 680-NHS dye (AF680; Thermo Fisher, InVitrogen™, Cat. No. A20008) on the N-terminus. Unmodified gold nanoparticles (SA-AuNP-SA) and Janus gold nanoparticles (SA-AuNP-SH) were reacted with AF680-G3-biotin separately. Following removal of free AF680-G3-biotin, the fluorescence intensity of each sample was acquired by Biotek Plate Reader Synergy HT. Both groups had higher fluorescence intensity over background fluorescence of unreacted gold nanoparticles, suggesting the successful modification of these particles with biotinylated ligands. The decrease in fluorescence intensity in the Janus gold nanoparticle group compared to unmodified gold nanoparticles indicated the reduced number of biotin-binding sites on the Janus particles. Similarly, AF680-Pep33-SMCC was reacted with unmodified gold nanoparticles (SA-AuNP-SA), Janus gold nanoparticles (SA-AuNP-SH) and biotin-ligand modified Janus gold nanoparticles (G3-AuNP-SH). The increase in fluorescence intensity indicated successful modification of the Janus particles with maleimide-ligands.

### Materials and Methods

### Preparation of Synthetic Particle Antibodies

Synthetic particle antibodies were prepared using solid phase synthesis. 100 mg aminomethyl chemmatrix resins after hydration with PBS were functionalized with 25 mg sulfo-NHS—S—S-biotin crosslinker (Apexbio Technology LLC, Houston, Tex.) in PBS at pH 7 in polypropylene reaction vessels at least 2 hours at 37° C. After extensive wash of the resin with PBS, 6-10e11 of streptavidin-coated gold nanoparticles of 30 nm in diameter (Nanohybrid. Inc, Austin, Tex.) was added into the vessel and incubated for at least 1 hour at 37° C. Bound gold nanoparticles were cleaved off from the resin by 2 ml 0.5M tris(2-carboxyethyl)phosphine) (TCEP) buffer to generate Janus gold nanoparticles with a streptavidin coated hemisphere and a thiol modified hemisphere. Following that, excessive TCEP buffer in the Janus nanoparticle solution was removed by centrifugal filtration with 100 KDa filter tubes (Amicon-ultra-4, Millipore-Sigma, US) at 700 g for 5 mins. These 100 KDa filter tubes were pretreated with 0.01% Tween-20 solution to reduce attaching of the particles onto the membranes. Fc-mimicking ligands Pep 33-maleimide were conjugated onto the thiol hemisphere of the Janus gold nanoparticles at pH 7.4 in PBS at room temperature followed by modification of the streptavidin hemisphere with an excess of biotinylated targeting ligands (e.g. G3-Biotin) in PBS. Unbound ligands were filtered out by centrifugal filters. Before treating particles to cells, the particle solutions were either filtered through 0.2 um filters or washed extensively with sterile buffer, such as PBS. To validate the presence of free thiols on the Janus gold nanoparticles, Alexa Fluor 647-maleimide dye was used instead of Pep33-SMCC in the first modification step followed by filtration and fluorescence measurement.

TEM Imaging of Janus Gold Nanoparticles with 3 nm Biotin-Gold Nanoparticles

2500 fold excessive amount of 3 nm biotin-gold nanoparticles (Nanocs. Inc, New York, N.Y.) was added to each types of gold nanoparticles (SA-AuNP-SA, SA-AuNP-SH, SA-AuNP-Pep33, G3-AuNP-G3) in PBS and reacted at room temperature for 2 hours. Then, the gold nanoparticle-biotin nanoparticle conjugates were centrifuged down at 4500 g for 30 mins. The conjugates were then resuspended in 20 ul of PBS. 10 ul of each type of conjugate solution was added onto separate TEM grids for TEM imaging. The grids were imaged with a Hitachi HT7700 transmission electron microscope.

### Characterization of Synthetic Particle Antibodies

The size of the MDSC targeting synthetic particle antibodies was characterized using Zeta-sizer (Malvern, USA). Validation of ligands modification on synthetic particle antibodies were conducted by fluorophore-labeled ligands and calibrated against a fluorescence standard curve. Briefly, ligands were first labeled with NHS ester-dye (Alexa Fluor 647 or Alexa Fluor 680). After filtering out the excessive dye, the labeled peptides were reacted with Janus nanoparticles following the modification procedure. After removing excessive fluorophore-labeled ligands, modified particles were used for fluorescence measurement.

### Example 2: In Vitro Testing of Synthetic Particle Antibodies

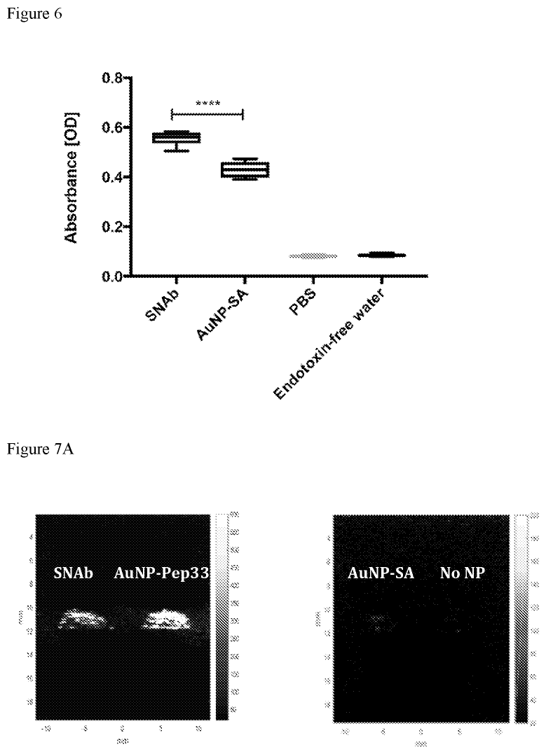

FIG. 6 showed that synthetic particle antibodies of the disclosure were capable of activating the NFkB proinflammatory pathway of RAW Blue macrophages. Activation of the NFkB inflammatory pathway, which is a key player in immune-regulation, is usually caused by Fc gamma receptor clustering and generally leads to the secretion of cytokines and inflammatory cellular activities. In each well of a 96-well plate, 100,000 RAW Blue macrophages (NFkB reporter cell line, InVivogen, US) were treated with 20 ul of different gold nanoparticle formulations of the same concentration (synthetic particle antibodies (“SNAb”), AuNP-SA), PBS and endotoxin-free water for 16 hrs. 50 microliters of supernatant was taken from each sample to determine the activation level of NFkB by Quanti-Blue substrate and absorbance measurement at 635 nm. The increase in the absorbance reading from wells treated with synthetic particle antibody groups indicated a higher level of activation of NFkB pathway, suggesting stronger immune activities of macrophages after treatment with synthetic particle antibodies.

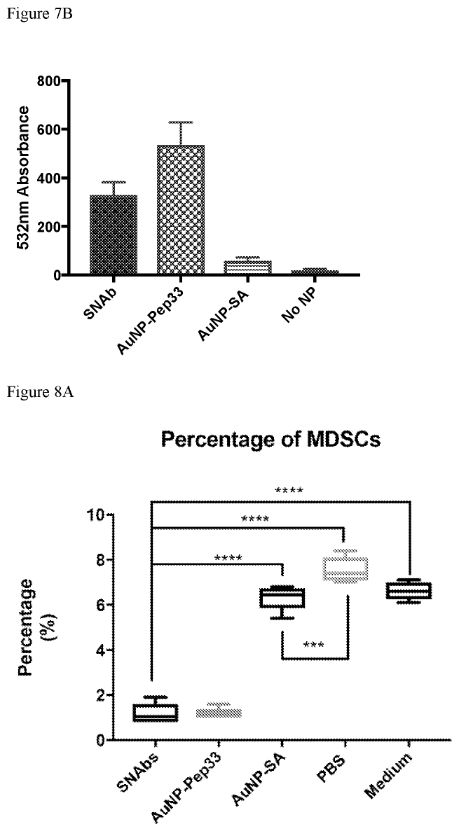

FIGS. 7A-7B demonstrate the ability of synthetic particle antibodies (“SNAbs”) of the disclosure to bind to cell targets by photoacoustic imaging. Myeloid derived suppressor cells (MDSCs) were used as an example cell target. MDSCs were isolated from 4T1-breast cancer bearing Balb/c mice and treated with same amount of different gold nanoparticle formulations (MDSC-SNAbs, AuNP-Pep33, unmodified non-Janus gold nanoparticles AuNP-SA) or PBS for 1 hr at 4° C. Cells were then washed to remove unbound nanoparticles and fixed. The cell samples were mixed with gelatin solution and formed domes on gelatin phantom. Photoacoustic signals (FIG. 7A) increased in the samples of SNAb or AuNP-Pep33 treated cell samples, indicating binding of these particles on these cells, possibly due to G3-MDSC interaction and Pep33-Fc receptor interaction (as MDSCs express Fc receptors). FIG. 7B showed particle abundance (bound on cells) in the samples of SNAb or AuNP-Pep33 treated cells.

### Materials and Methods

### Photoacoustic Imaging of Ligands-Modified Gold Nanoparticles Binding on MDSCs

Synthetic particle antibodies, Pep33-modified gold nanoparticles were fabricated according to the protocol described above. 1.5 million of MDSCs were seeded in each well of a 24 well plate and treated with 1e11 of nanoparticles. After 1 hour of incubation at 4° C., cells were collected into microcentrifuge tubes and washed with PBS. Cells were then fixed with BD Cytofix buffer and washed again 3 times. Samples were resuspended into 40 ul of PBS buffer and before imaging, they were mixed with hot 16% gelatin solution and then solidified into a dome on a gelatin phantom at 4° C. The domes were imaged with Vevo 2100/LAZR system made by FUJIFILM Visual Sonics.

### NFkB Proinflammatory Pathway Activation of RAW Blue Cells by Synthetic Particle Antibodies

100,000 RAW Blue macrophages (InvivoGen, San Diego, Calif.) were plated in a flat bottom 96 well plate in 180 ul test medium and treated with 20 ul of synthetic particle antibodies, AuNP-SA, PBS or endotoxin-free water. Co-cultures were incubated at 37° C. for 20 hrs and 50 ul of supernatants from each well were harvested for analysis of NFkB activation with the 150 ul Quanti-blue substrate. After 60 min incubation, the plate was read at 635 nm for absorbance.

### Example 3: In Vivo Testing of Synthetic Particle Antibodies

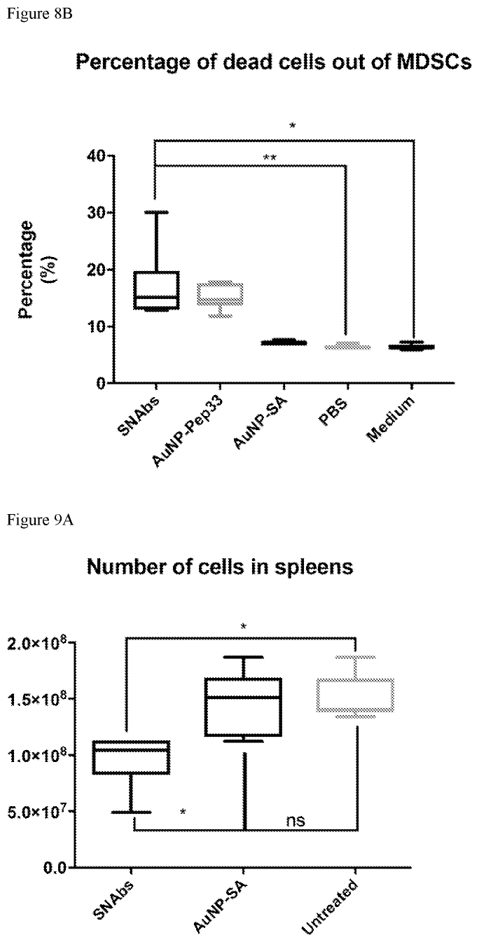

FIGS. 8A-8B show that synthetic particle antibodies (“SNAbs”) can induce killing of MDSCs in splenocyte mixed co-cultures. Splenocytes single-cell suspension from 4T1-breast cancer bearing Balb/c mice were treated with the same amount of gold nanoparticle formulations (G3-AuNP-Pep33, i.e., MDSC-SNAb, AuNP-Pep33, AuNP-SA), PBS or medium for 24 hrs. The cell mixture was then stained with antibodies against CD11b and Gr-1 for MDSCs, with propidium iodide for dying cells, and analyzed with BD Fortessa flow cytometer. The lower percentage of MDSCs (FIG. 8A) and higher percentage of dying cells in the MDSC population (FIG. 8B) in the SNAb and AuNP-Pep33 treated sample indicated that cell lysis was being triggered by SNAbs against MDSCs.

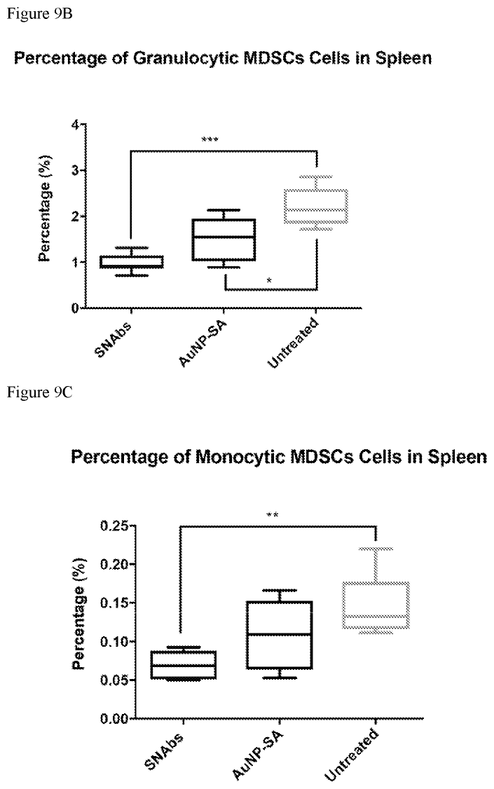

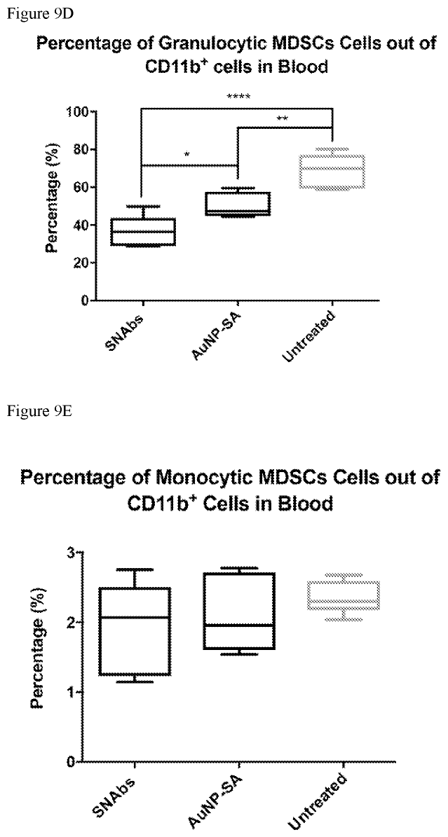

FIGS. 9A-9E show in vivo depletion of MDSCs by synthetic particle antibodies (“SNAbs”) in a 4T1 breast cancer murine model. 4T1 breast cancer-bearing Balb/c mice were treated with 7.5e+10 nanoparticles (SNAbs, or AuNP-SA) in 200 ul of PBS on day 10 post tumor inoculation or left-untreated. After 24 hrs, the spleens, blood and tumors were collected from the mice and analyzed for different cell populations (MDSCs, CD3+CD4+ T cells, CD3+CD8+ T cells, CD25+Foxp3+ T cells, NK cells, B cells) by flow cytometry. A decrease in the total number of cells in the spleens in the SNAb-treated group implied the amelioration of splenomegaly caused by tumors (FIG. 9A). A reduction in the percentages of granulocytic MDSCs and monocytic MDSCs in the spleen (FIGS. 9B and 9C, respectively) and blood (FIGS. 9D and 9E, respectively), which are the two major organs where MDSCs reside, also reflected the therapeutic effect of target-cell depletion by SNAbs in vivo.

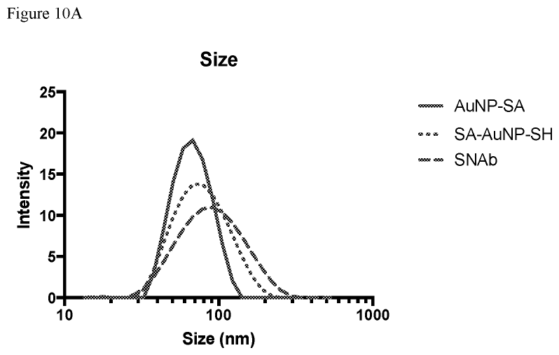

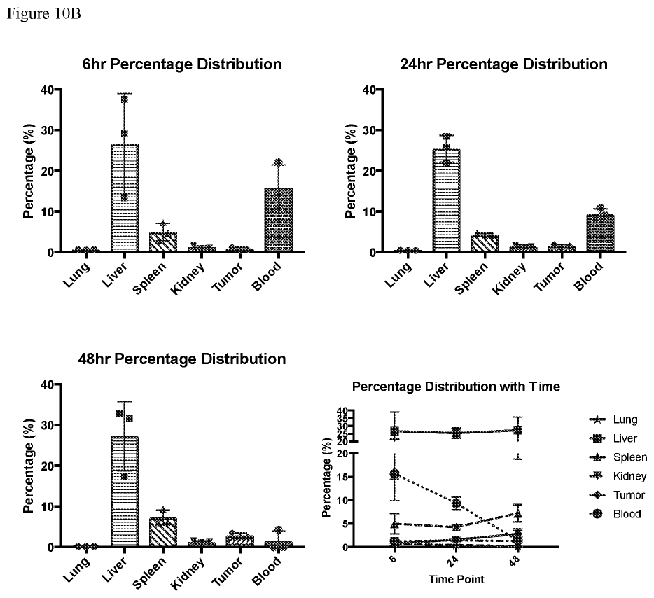

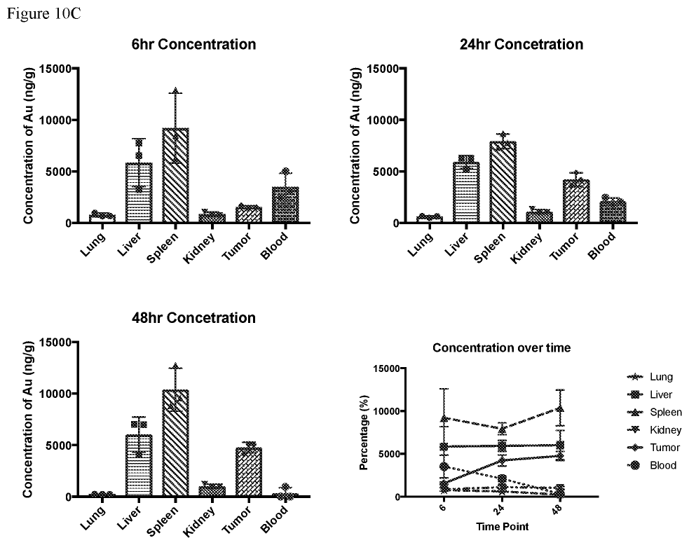

FIGS. 10A-10C show in vivo distribution of synthetic particle antibodies (“SNAbs”) in lung, liver, spleen, kidney, tumor and blood in a 4T1 breast cancer murine model. (FIG. 10A) Size of non-Janus AuNP-SA, Janus SH-AuNP-SA, SNAbs as determined by zetasizer. Particle hydrodynamic size increased from about 70 nm to about 100 nm after Janus particle fabrication and modification with ligands. (FIG. 10B) Biodistribution of SNAbs in different organs by percentage at different time points after intravenous injection via tail vein in 4T1-breast tumor bearing Balb/c mice. The biodistribution is calculated as the percentage of Au in each organ out of the sum of the amount measured in the six organs, showing relative abundancy of synthetic particle antibodies in each of these organs. The majority of the injected SNAbs went to the liver and remained in circulation at t=6 hrs. The accumulation of SNAbs in circulation dropped sharply and increased in tumors over time. (FIG. 10C) Biodistribution of SNAbs in different organs by concentration at different time points after intravenous injection via tail vein in 4T1-breast tumor bearing Balb/c mice. The concentration of gold in both spleen and tumor were very high compared to other organs over time, indicating positive therapeutic potential of SNAbs in vivo. The concentration of gold dropped in blood from 6 hr to 48 hr, suggesting a half-life around 26 hrs. As expected for nano-sized gold nanoparticles, the liver had a significant accumulation of Au. The biodistribution of the bi-functional particles thus can be altered with different surface chemistry and size of the particles.

### Materials and Methods

### Cell Lines, Mice, and Tumor Growth In Vivo

For the experiments, 4T1 and RAW 264.7 mouse macrophage-like cell line were purchased from American Type Culture Collection (Manassas, Va., USA). The tumor cell lines were cultured in RPMI 1640 (Thermo Fisher Scientific, Waltham, Mass., USA), while RAW 264.7 cell line was cultured in DMEM (Thermo Fisher Scientific), both supplemented with 10% FBS (Hyclone GE) and 1% Penicillin-Streptomycin (Thermo Fisher Scientific) under standard cell culture condition (37° C., 5% CO₂).

Five to six-week-old Balb/c female mice were purchased from the Jackson Lab. All mice were maintained in a pathogen-free mouse facility according to institutional guidelines. All the animal experiments were approved by the Institutional Animal Care and Use Committee (IACUC) at Georgia Institute of Technology (Atlanta, Ga.). The experimental sample sizes, which included all of the mice, ensured adequate statistical power. But the experiments did not entail randomization and blinding.

For tumor generation, single cell suspension of 4T1 before passage 25 were prepared in PBS (Hyclone) at a concentration of 1×10⁷ cells/mL. Balb/c mice were inoculated with 0.5×10⁶ 4 T1 breast cancer cells in 50 ul sterile PBS orthotopically at the fourth mammary fat pad on Day 0. For Myeloid-derived suppressor cell (MDSC) isolation, tumor-bearing mice were killed between day 12 to 30 after a decent-sized (>5 mm) primary tumor was established. For in vivo studies, tumor-bearing mice were killed when any of the following symptoms appear: (1) subcutaneous tumor burden reaches 1.5 cm in any direction; (2) ulceration or bleeding of tumors; (3) ruffled fur coat; (4) disability in moving or difficulties in intake of food and water; (5) excessive abdominal distension and diarrhea; and/or (6) appearance of cachexia including severe weight loss.

Isolation of MDSC from the Spleens of Tumor-Bearing Mice

Balb/c mice were inoculated with 0.5×10⁶ 4 T1 breast cancer cells on Day 0. Spleens were harvested after 10-20 days from tumor-bearing animals and minced into thin pieces followed by dissociation in collagenase D (2 mg/ml) in RPMI 1640 medium for 0.5-1 hr at room temperature. Dissociated spleen tissues were then passed through a 40-μm nylon cell strainer (CellTreat. Inc, Pepperell, Mass.) to obtain single cell suspension. Red blood cells were lysed in 1×lysis buffer (BD Bioscience, US). Gr1⁺ MDSC cells were isolated from the RBC-lysed single cell suspension by magnetic cell sorting using the mouse MDSC isolation kit, according to the manufacturer's protocol (Miltenyi Biotec, Auburn, Calif., USA).

### Splenocyte Killing of MDSCs Triggered by Targeting SNAbs

Spleens were harvested after 10-20 days from tumor-bearing animals and minced into thin pieces followed by dissociation in collagenase (2 mg/ml; Roche Diagnostics GMbH, Mannheim, Germany) in RPMI 1640 (Thermo Fisher Scientific) for 0.5-1 hr at room temperature. Dissociated spleen tissues were then passed through a 40-μm nylon cell strainer (CellTreat) to obtain single cell suspension. RBCs were lysed in 1×lysis buffer (BD PharMingen-US). 1×10⁶ cells were distributed into each well in 96 well plate in 200 ul of RPMI 1640 medium. G3-AuNP-Pep33 or AuNP-Pep33 or AuNP-SA formulation in 100 ul sterile PBS was dispensed into the corresponding wells respectively. Control wells were treated with sterile PBS or RPMI 1640 complete medium. After 24 hrs of 37° C. incubation, cells were harvested for flow cytometry analysis using BD LSRFortessa. Antibodies used for cell marker staining includes anti-F4/80-FITC, anti-CD11c-PE, anti-B220-FITC, anti-CD8-FITC, anti-CD4-PE, anti-CD3-PE-Cy7, anti-CD11b-PE-Cy7, anti-Ly6G-PerCP-Cy5.5, anti-Ly6C-APC-Cy7, anti-CD49b-APC, anti-FoxP3-APC and anti-CD25-APC-Cy7.

### In Vivo Depletion of MDSCs by Synthetic Particle Antibodies

Mice of age 5-12 weeks were inoculated orthogonally with 4T1 breast cancer cells 0.5×10⁶/50 ul of sterile PBS on Day 0. 200 ul of G3-AuNP-Pep33 synthetic particle antibody formulation or unmodified streptavidin functionalized gold nanoparticles (AuNP-SA) formulation containing 7.5×10¹⁰ nanoparticles were administrated intravenously through tail vein injection to mice (n=6). After 24 hrs, mice were euthanized. Spleens and tumors were collected and processed as described previously. Briefly, spleens and tumors were treated with collagenase D and RBC lysis to single cell suspensions. Blood was also collected from the mice by cardiac puncture. 150 ul of blood from each mouse was transferred to fresh FACS tubes and treated with 2 ml RBC lysis buffer at room temperature for 10 minutes and then centrifuged at 700 g for 10 mins. The supernatant was discarded. RBC lysis was repeated once more and the cell pellets were resuspended in FACS buffer. Spleen, tumor and blood cells were stained with antibodies for MDSC (Ly6G, Ly6C), macrophages (F4/80), T cells (CD3, CD4, CD8, Foxp3, CD25), B cells and NK cells (CD49b, CD3, CD335). Data was collected with BD LSRFortessa for identification of various cell types.

### In Vivo Biodistribution

8e10 synthetic particle antibodies in 200 ul PBS were injected intravenously via tail vein into Balb/c mice on day 9 post 4T1 tumor inoculation (as described above). Three mice were euthanized at 6 hr, 24 hr, and 48 hr each after injection. Lung, liver, kidney, spleen, tumor, and blood were collected from each mouse, weighed and dissolved in aqua regia solution (prepared by mixing concentrated nitric acid:hydrochloride acid in 1:3 volume ratio; nitric acid was purchased from Sigma Cat. #695025; hydrochloride acid was purchased from VWR International, Cat #. BDH3030). The samples were incubated in aqua regia overnight and then boiled at 200° C. to further dissolve the tissues and gold particles as well as to remove aqua regia. Then samples were resuspended in 3 ml of deionized water and passed through a 0.2 um filter. The concentration of Au in each sample was measured using inductively coupled plasma-mass spectrometer (ICP-MS) and converted to the concentration of synthetic particle antibodies in each organ.

### Example 4: Use of Synthetic Particle Antibodies to Treat and/or Prevent Various Diseases and/or Conditions

In some embodiments, the synthetic particle antibodies of the disclosure can be used to specifically target and deplete immune suppressor cells and/or cancer cells. In such embodiments, the synthetic particle antibodies are engineered with targeting ligands against immune suppressor cells and/or cancer cells. In some embodiments, the immune-activating ligands on the opposite face of the synthetic particle antibody can bind to Fc receptors on immune cells and facilitate antibody-dependent cell killing, such as for example and not limitation, use of Pep33 peptides to target and deplete myeloid-derived suppressor cells as shown in more detail herein. Other targeting ligands contemplated by the disclosure can target the synthetic particle antibodies to other immune suppressor cells (e.g., regulatory T-cells), to well-studied and validated cancer specific targets (e.g. CD33, HER2, CD52, CD20, EGFR), and/or to novel disease-specific targets that can be identified using phage display or other methods as discussed herein.

In further embodiments related to treating and/or preventing cancer, the synthetic particle antibodies of the disclosure can be used in combination with other cancer therapies as discussed herein, such as for example and not limitation, with cancer vaccines, chemotherapeutics, and radiation-based chemotherapy. Without wishing to be bound by theory, it is suggested that cancer vaccine efficacy can often be limited by the presence of checkpoint blockade and immune suppressor cells, which can thus limit the extent of the immune response in the tumor. The synthetic particle antibodies of the disclosure could be used to deplete myeloid-derived suppressor cells (MDSCs) and/or tumor-associated macrophages (TAMs), thereby possibly removing one mechanism of immune suppression and subsequently enhancing the immunogenicity of the cancer vaccine. In some embodiments, the synthetic particle antibodies of the disclosure (e.g., polymer-based particles) could be designed to encapsulate chemotherapeutics within the particle core, and/or the synthetic particle antibodies could be delivered in combination with existing chemotherapy regimens. One challenge with chemotherapy is the existence of intracellular resistance mechanisms that hinder therapeutic efficacy. The synthetic particle antibodies of the disclosure can enable killing by two mechanisms (antibody-mediated and chemotherapy mediated), which can enhance overall therapeutic efficacy. The synthetic particle antibodies of the disclosure can be delivered in conjunction with radiation therapy for cancer patients. While radiation therapy is successful at inducing apoptosis in tumors, it also forms an environment that is favorable for the proliferation of regulatory T cells that can negate the anti-tumor effect. Gold particle-based synthetic particle antibodies can be used to deplete immune-suppressor cells (e.g., MDSCs) and at the same time assist phototherapy for cancer destruction, as a result of which an immune-promoting environment is created for T cell to eliminate tumor cells. Targeted depletion of regulatory T cells could enable improved outcomes with radiation therapy. Alternatively, synthetic particle antibodies of the disclosure could be engineered to target tumor cells that have upregulated ligands facilitating checkpoint blockade (e.g., PD-L1) to promote an anti-tumor effect.

In other embodiments, synthetic particle antibodies of the disclosure can be engineered with targeting ligands that recognize T cells in subjects with autoimmune diseases, such as for example and not limitation, systemic lupus erythematosus (SLE), and can thus treat and/or prevent the autoimmune disease by depleting such T cells. In other embodiments, synthetic particle antibodies of the disclosure can be engineered with targeting ligands to specifically detect idiotypes on autoantibodies to deplete B cells that recognize the same auto-antigen, and thus can also be used to treat and/or prevent the autoimmune disease by depleting such B cells. In still other embodiments, specifically for treating and/or preventing multiple sclerosis (MS), synthetic particle antibodies of the disclosure can be engineered with targeting ligands to recognize specific integrins (e.g., integrin α-4) on the surface of T cells, which can prevent T cell proliferation into central nervous system (CNS) lesions. Other MS-specific therapies that are contemplated by the disclosure include the use of synthetic particle antibodies of the disclosure can be engineered with targeting ligands to specifically detect and deplete monocytes and lymphocytes in the bloodstream, and in further embodiments can be used to treat and/or prevent relapsing-remitting MS.

In other embodiments, synthetic particle antibodies of the disclosure can be engineered with targeting ligands that specifically recognize viruses, bacteria, parasites, fungi, and other disease-causing microorganisms.

In other embodiments, synthetic particle antibodies of the disclosure can be engineered with targeting ligands that specifically recognize and bind to the IL-2 receptor on T-cells, which could prevent T-cell activation and subsequent B-cell activation in kidney transplant recipients.

In other embodiments, synthetic particle antibodies of the disclosure can be engineered with targeting ligands that specifically recognize TNF-alpha, IL-12, or IL-23, all of which are cytokines that lead to severe inflammation in inflammatory bowel disease (IBD).

In other embodiments, synthetic particle antibodies of the disclosure can be engineered with targeting ligands that specifically recognize IL-17a and TNF-alpha, both of which are cytokines that are implicated in psoriasis.

In other embodiments, synthetic particle antibodies of the disclosure can be engineered with targeting ligands that specifically recognize TNF-alpha and/or IL-4, both of which are cytokines that are implicated in GVHD.

In any of the above embodiments, the size and/or shape of the synthetic particle antibody can be modified based on the target tissue, organ, and/or disease or condition being treated and/or prevented as discussed in more detail herein.

### Example 5: Use of Synthetic Particle Antibodies in Diagnostic Applications

In some embodiments, synthetic particle antibodies of the disclosure can be formulated using a particle core that also serves as a contrast agent. The particle core can function as a contrast agent by, for example and not limitation, being a contrast agent itself (e.g., a metal or metal oxide particle), having a contrast agent encapsulated in the particle itself, and/or having the contrast agent functionally attached to the particle. The contrast agent enables the synthetic particle antibodies to detect cell targets determined by the targeting ligands on the opposite surface of the bi-functional particle. Non-limiting examples of particle contrast agents include iron oxide nanoparticles for MRI imaging or gold nanoparticles for x-ray computed tomography or photoacoustic imaging.

### Example 6: Use of Synthetic Particle Antibodies in Research Applications

In some embodiments, the synthetic particle antibodies of the disclosure can be used in various research applications involving antibodies, such as for example and not limitation, immunoprecipitation, immunohistochemistry, and/or immunoblotting. It is intended that the synthetic antibodies of the disclosure can replace non-synthetic antibodies in these applications.

When used in immunoprecipitation applications such as for example and not limitation, pull-down assays and column-based purification, the synthetic particle antibodies of the disclosure can be engineered with targeting ligands on one face and immune-activating ligands on the opposite face that can be recognized by a bead (e.g., agarose, iron oxide, polypropylene gel), which allows the separation of antibody-antigen complexes by size and/or affinity to the receptor on the bead. Depending on the size of the core nanoparticle, the whole construct could also facilitate a one-step method to separate antibody-antigen complexes. In another embodiment, the agarose

When used in immunohistochemistry applications such as for example and not limitation, antigen staining in a tissue of interest, the synthetic particle antibodies of the disclosure can be engineered with targeting ligands on one face and immune-activating ligands on the opposite face that can be recognized by a secondary fluorescent and/or radioactive antibody. The secondary antibody can enable the use of the synthetic particle antibodies for staining tissue for histological sections.

When used in immunoblotting applications such as for example and not limitation, Western blotting and enzyme-linked immunosorbent assays, the synthetic particle antibodies of the disclosure can be engineered with targeting ligands on one face and immune-activating ligands on the opposite face that can be recognized by a secondary fluorescent and/or radioactive antibody. The secondary antibody can enable the use of the synthetic particle antibodies for detecting binding of the synthetic particle antibody to a target of interest.

While several possible embodiments are disclosed above, embodiments of the present disclosure are not so limited. These exemplary embodiments are not intended to be exhaustive or to unnecessarily limit the scope of the disclosure, but instead were chosen and described in order to explain the principles of the present disclosure so that others skilled in the art may practice the disclosure. Indeed, various modifications of the disclosure in addition to those described herein will become apparent to those skilled in the art from the foregoing description. Such modifications are intended to fall within the scope of the appended claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

All patents, applications, publications, test methods, literature, and other materials cited herein are hereby incorporated by reference in their entirety as if physically present in this specification.

### REFERENCES

Peiris P M, Schmidt E, Calabrese M, Karathanasis E. Assembly of Linear Nano-Chains from Iron Oxide Nanospheres with Asymmetric Surface Chemistry. (2011) PLoS ONE 6(1): e15927. doi:10.1371/journal.pone.0015927.
Saifullah L, Cheong In Woo. Fabrication of Polymeric Janus Particles by Droplet-Microfluidics. (2014) RSC Adv., 2014, 4, 13322-13333.
Zhihong Nie, Li Wei, Seo Minseok, Xu Shengqing, and Kumacheva Eugenia. Janus and Ternary Particles Generated by Microfluidic Synthesis: Design, Synthesis, and Self-Assembly. (2006) J. Am. Chem. Soc., 128, 9408-9412
Jing Hu, Shuxue Zhou, Yangyi Sun, Xiaosheng Fang and Limin Wu. Fabrication, properties and applications of Janus particles. (2012) Chem. Soc. Rev., 41, 4356-4378.
Tang Jennifer L., Schoenwald Kipp, Potter Daniel, White David, and Sulchek Todd. Bifunctional Janus Microparticles with Spatially Segregated Proteins. (2012) Langmuir, 28, 10033-10039.
Garbuzenko Olga B., Winkler Jennifer, Tomassone M. Silvina, and Minko Tamara. Biodegradable Janus Nanoparticles for Local Pulmonary Delivery of Hydrophilic and Hydrophobic Molecules to the Lungs. (2014 Nov. 4) Langmuir.; 30(43): 12941-12949.
Wang, A. Z. & Farokhzad, O. C. Current progress of aptamer-based molecular imaging. (2014) J. Nucl. Med. 55, 353-356.
Bonetto Stephane, Spadola Loredana, Buchanan Andrew G., Jermutus Lutz, and J. L. Identification of cyclic peptides able to mimic the functional epitope of IgG1-Fc for human FcγRI. (2009) FASEB J. 23, 575-585.
Mcenaney, P. J. et al. Chemically Synthesized Molecules with the Targeting and Effector Functions of Antibodies. (2014) J. Am. Chem. Soc. 136, 18034-18043.
Choi C H J, Zuckerman J E, Webster P, Davis M E Targeting kidney mesangium by nanoparticles of defined size. (2011) Proc Natl Acad Sci USA 108(16):6656-6661.
Shilo M, Reuveni T, Motiei M, Popovtzer R. Nanoparticles as computed tomography contrast agents: Current status and future perspectives. (2012) Nanomedicine (Lond) 7(2):257-269.
Moghimi S M, Hunter A C, Murray J C. Nanomedicine: Current status and future prospects. (2005) FASEB J 19(3):311-330.
Dreaden, EC, Austin Lauren A, Mackey Megan A, and El-Sayed Mostafa A, Size matters: gold nanoparticles in targeted cancer drug delivery. (2012 April) Ther Deliv. 3(4): 457-478.
Tang L. et al. Investigating the optimal size of anticancer nanomedicine. (2014) Proc. Natl. Acad. Sci. 111, 15344-15349.
Ellis S E, Newlands G F, Nisbet A J, Matthews J B. Phage-display library biopanning as a novel approach to identifying nematode vaccine antigens. (2012 May) Parasite Immunol. 34(5):285-95.
Molek P, Strukelj B, Bratkovic T. Peptide phage display as a tool for drug discovery: targeting membrane receptors. (2011 January) Molecules 21; 16(1):857-87.
Zhou J, Rossi J. Aptamers as targeted therapeutics: current potential and challenges. (2017 March) Nat Rev Drug Discov. 16(3):181-202.
Wang, A. Z. and Farokhzad, 0. C. Current progress of aptamer-based molecular imaging. (2014) J. Nucl. Med. 55, 353-356.
Scott A M, Wolchok J D, Old L J. Antibody therapy of cancer. (2012 March) Nat Rev Cancer. 22; 12(4):278-87.
Dorresteijn, B. Nanobody-based cancer therapy of solid tumors. (2015) Nanomedicine (Lond.) 10, 161-174.
Holliger, P., Hudson, P. J. Engineered antibody fragments and the rise of single domains. (2005) Nat. Nanotechnol. 23, 1126-1136.
De Meyer, T., Muyldermans, S., Depicker, A. Nanobody-based products as research and diagnostic tools. (2014) Trends Biotechnol. 32, 263-270.
Danquah, W. et al. Nanobodies that block gating of the P2X7 ion channel ameliorate inflammation. (2016) Sci. Transl. Med. 8.
Secchiero, P. et al. Selection and characterization of a novel agonistic human recombinant anti-Trail-R2 minibody with anti-leukemic activity. (2009) Int. J. Immunopathol. Pharmacol. 22, 73-83.
Wu, B., Sun, Y. Pharmacokinetics of Peptide-Fc Fusion Proteins. (2014) J. Pharm. Sci. 103, 53-64.
Wesolowski, J. et al. Single domain antibodies: promising experimental and therapeutic tools in infection and immunity. (2009 August) Med Microbiol Immunol. 198(3):157-74.
Safenkova Irina V., Anatoly V. Zherdev, Boris B. Dzantiev. Correlation between the composition of multivalent antibody conjugates with colloidal gold nanoparticles and their affinity. Journal of Immunological Methods 357 (2010) 17-25.
Soukka Tero, Harri Harma, Janika Paukkunen, and Timo Lovgren. Utilization of Kinetically Enhanced Monovalent Binding Affinity by Immunoassays Based on Multivalent Nanoparticle-Antibody Bioconjugates. Anal. Chem. 2001, 73, 2254-2260.
Choi Jeong-Woo, Da-Yeon Kang, Yong-Hark Jang, Hyun-Hee Kim, Junhong Min, Byung-Keun Oh. Ultra-sensitive surface plasmon resonance based immunosensor for prostate-specific antigen using gold nanoparticle-antibody complex. Colloids and Surfaces A: Physicochem. Eng. Aspects 313-314 (2008) 655-659.
Jung Su-Na, Sang-Kee Kang, Guen-Hye Yeo, Hai-Ying Li, Tao Jiang, Jae-Woon Nah, Jin-Duck Bok, Chong-Su Cho, Yun-Jaie Choi. Targeted Delivery of Vaccine to Dendritic Cells by Chitosan Nanoparticles Conjugated with a Targeting Peptide Ligand Selected by Phage Display Technique. Macromol. Biosci. 2014, DOI: 10.1002/mabi.201400352.
Gray Bethany Powell, Shunzi Li, and Kathlynn C. Brown. From Phage Display to Nanoparticle Delivery: Functionalizing Liposomes with Multivalent Peptides Improves Targeting to a Cancer Biomarker Bioconjugate Chem. 2013, 24, 85-96.
Kaewsaneha Chariya, Pramuan Tangboriboonrat, Duangporn Polpanich, Mohamed Eissa, and Abdelhamid Elaissari. Janus Colloidal Particles: Preparation, Properties, and Biomedical Applications ACS Appl. Mater. Interfaces 2013, 5, 1857-186.
Torchia James, Kipp Weiskopf and Ronald Levy. Targeting lymphoma with precision using semisynthetic anti-idiotype peptibodies. PNAS 2016, 113 (19), 5276-5381.
Ortiz Daniel F. et al., Elucidating the interplay between IgG-Fc valency and FcgR activation for the design of immune complex inhibitors. Sci. Transl. Med, 2016, 8 (365).
Anon, 2017. Custom Mouse Monoclonal Antibody Production Protocols. Thermo-Fisher Scientific, p. 1. Available at: https://www.thermofisher.com/us/en/home/life-science/antibodies/custom-antibodies/custom-antibody-production/custom-monoclonal-antibody-production/custom-mouse-monoclonal-antibody-production-protocols.html.
Anon, 2016. Monoclonal Antibodies (mAbs) Market Analysis By Source (Chimeric, Murine, Humanized, Human), By Type of Production, By Indication (Cancer, Autoimmune, Inflammatory, Infectious, Microbial, Viral Diseases), By End-use (Hospitals, Research, Academic Institut. Market Research Report, p. 115.
Bru, M. et al., 2015. Human Antibody Production in Transgenic Animals, Arch Immunol Ther Exp (Warsz). 2015; 63(2): 101-108.
Cabral, H. et al., 2015. Systemic Targeting of Lymph Node Metastasis through the Blood Vascular System by Using Size-Controlled Nanocarriers. ACS Nano, 2015, 9(5), pp 4957-4967.
Cai, H. H., 2017. Therapeutic Monoclonal Antibodies Approved by FDA in 2016. MOJ Immunol, 5(1), pp. 2016-2017.
Chames, P. et al., 2009. Therapeutic antibodies: Successes, limitations and hopes for the future. British Journal of Pharmacology, 157(2), pp. 220-233.
Popov Jesse, Anita I Kapanen, Christopher Turner, Rebecca Ng, Catherine Tucker, Gigi Chiu, Richard Klasa, M. B. B. & Chikh, & G., 2011. Multivalent rituximab lipid nanoparticles as improved lymphoma therapies: indirect mechanisms of action and in vivo activity. nanomedicine, 6(9), pp. 1575-1592.
Matsumoto, Y. et al., 2016. Vascular bursts enhance permeability of tumour blood vessels and improve nanoparticle delivery. Nature Nanotechnology, 11(6), pp. 533-538. Available at: http://dx.doi.org/10.1038/nnano.2015.342.
Mazor, Y. et al., 2017. Enhanced tumor-targeting selectivity by modulating bispecific antibody binding affinity and format valence. Nature Publishing Group, (October 2016), pp. 1-11. Available at: http://dx.doi.org/10.1038/srep40098.
Montet, X. et al., 2006. Multivalent effects of RGD peptides obtained by nanoparticle display. Journal of Medicinal Chemistry, 49(20), pp. 6087-6093.
Schirrmann, T. & Hust, M., 2016. Phage display-derived human antibodies in clinical development and therapy. MABS, 8(7), pp. 1177-1194.
Shaughnessy, A. F., 2012. Monoclonal antibodies: magic bullets with a hefty price tag. BMJ, 8346 (December), pp. 10-12.
Wang, Y. & Fan, Z., 2016. Nanobody-derived nanobiotechnology tool kits for diverse biomedical and biotechnology applications. International Journal of Nanomedicine, 11, pp. 3287-3303.
Zhang, Y., Hoppe, A. D. & Swanson, J. A, 2010. Coordination of Fc receptor signaling regulates cellular commitment to phagocytosis. PNAS, 107(45), pp. 19332-19337.

1. A synthetic particle antibody composition comprising:
a bi-functional particle core that has at least two different surface chemistries;
a targeting ligand conjugated to one of the surface chemistries of the bi-functional particle core; and
an immune-activating ligand conjugated to another of the surface chemistries of the bi-functional particle core.

2. The synthetic particle antibody composition of claim 1, wherein the bi-functional particle core comprises a Janus particle.

3. The synthetic particle antibody composition of claim 1, wherein the targeting ligand is selected from the group consisting of a protein, a peptide, an aptamer, and fragments thereof.

4. The synthetic particle antibody composition of claim 1, wherein the immune-activating ligand is selected from the group consisting of a fragment of the Fc portion of antibodies, an immune-activating peptide, and proteins or peptides that mimic the structure and/or function of the Fc portion of antibodies.

5. The synthetic particle antibody composition of claim 1, wherein the targeting ligand comprises the G3 peptide; and
wherein the immune-activating ligand comprises the Pep33 peptide.

6. A synthetic particle antibody composition comprising:
a bi-functional particle core that has at least two different surface chemistries;
at least one targeting ligand conjugated to one of the surface chemistries of the bi-functional particle core, wherein one or more targeting ligand has the ability to specifically bind to a desired cell or tissue type in a patient's body; and
at least one immune-activating ligand conjugated to another of the surface chemistries of the bi-functional particle core.

7. A therapeutic method of treating cancer in a patient in need thereof comprising:
administering a therapeutically effective amount of the synthetic particle antibody composition of claim 6;
wherein at least one targeting ligand has specificity to a target selected from the group consisting of a tumor-associated antigen characteristic of the cancer being treated, and a cell surface molecule expressed by a MDSC or a regulatory T cell.

8. A therapeutic method of treating an autoimmune disease in a patient in need thereof comprising:
administering a therapeutically effective amount of the synthetic particle antibody composition of claim 6;
wherein at least one targeting ligand has specificity to a target selected from the group consisting of a molecule characteristic of the autoimmune disease being treated, a surface molecule expressed by a cell that is a cause of the autoimmune disease or produces the deleterious symptoms of the disease, and a molecule that is implicated as a cause of an effect of the autoimmune disease.

9. A therapeutic method of treating an infection in a patient in need thereof comprising:
administering a therapeutically effective amount of the synthetic particle antibody composition of claim 6;
wherein the infection being treated is selected from the group consisting of bacterial, viral, parasitic, and fungal; and
wherein at least one targeting ligand has specificity to a target selected from the group consisting of an antigen characteristic of the infection being treated, and a cell surface molecule expressed by a MDSC or a regulatory T cell.

10. A method of diagnosing a disease or condition in a subject comprising:
obtaining a bodily fluid or tissue sample from the subject;
contacting the sample with the synthetic particle antibody composition of claim 6; and
determining the presence or absence of an antigen that is characteristic of the disease or condition.

11. A method of performing in vivo imaging in a patient in need thereof comprising:
administering the synthetic particle antibody composition of claim 6;
placing the patient in an appropriate imaging machine suitable for contrast imaging; and
performing the contrast imaging;
wherein the synthetic particle antibody composition further comprises a contrast agent comprising iron oxide particles or gold particles.

12. A method of immunoprecipitation comprising:
mixing and incubating a sample lysate with the synthetic particle antibody composition of claim 6, wherein the synthetic particle antibody composition is conjugated to an antigen of interest;
mixing the sample lysate and synthetic particle antibody composition with at least one suitable bead for immunoprecipitation; and
washing and eluting the sample lysate from at least one bead.

13. A method of immunohistochemistry comprising:
fixing a tissue sample in 4% formaldehyde solution;
embedding the fixed tissue sample in either tissue freezing medium or paraffin;
slicing the embedded tissue sample in 10-20 μm sections;
adding an appropriate blocking solution to the sliced tissue section;
adding the synthetic particle antibody composition of claim 6 to the tissue section;
adding a secondary antibody composition that recognizes the immune-activating ligands on the synthetic particle antibody composition of claim 6 to the tissue section;
washing and mounting the tissue sections; and
imaging the washed and mounted tissue sections for microscopy.

14. A method of enzyme-linked immunosorbent assay (ELISA) comprising:
coating a well plate or other substrate with the synthetic particle antibody composition of claim 6;
adding a sample with proteins that are recognized by at least one targeting ligands on the synthetic particle antibody composition;
adding a secondary antibody composition that recognizes at least one immune-activating ligands on the synthetic particle antibody composition of claim 6, wherein the secondary antibody composition is conjugated to at least one reporter selected from the group consisting of a fluorophore, a chemiluminescent substrate, a radioactive label, and a tertiary antibody linked to an enzyme; and
performing an assay measuring fluorescence from the secondary antibody composition or absorbance from reaction of the tertiary antibody linked to an enzyme with a substrate.

15. A method of immunoblotting comprising:
isolating proteins from tissue samples or cell culture;
separating proteins using gel electrophoresis;
transferring proteins from the gel to a membrane;
blocking the membrane to prevent non-specific interactions with proteins and the synthetic particle antibody composition of claim 6;
incubating the membrane with the synthetic particle antibody composition with targeting ligands specific to a protein of interest;
rinsing the membrane and adding a secondary antibody composition that recognizes at least one immune-activating ligands on the synthetic particle antibody composition of claim 6, in which the secondary antibody can be composition is conjugated to at least one reporter selected from the group consisting of a fluorophore, a chemiluminescent substrate, a radioactive label, and a tertiary antibody linked to an enzyme; and
performing an assay that measures protein levels by methods that are not limited to one or more of fluorescence, luminescence, and radiography.
